(19) United States
(12) Patent Application Publication
(10) Pub. No.: US 2003/0235854 A1
(43) Pub. Date: Dec. 25, 2003

(54) Methods for analyzing a nucleic acid

(76) Inventors: Eugene Y. Chan, Brookline, MA (US); Eric Nalefski, Woburn, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/435,399

(22) Filed: May 9, 2003

Related U.S. Application Data
(60) Provisional application No. 60/379,461, filed on May 9, 2002.

(51) Int. Cl. C12Q 1/68

(52) U.S. Cl. 435/6

(57) ABSTRACT

Disclosed herein are methods for analyzing a nucleic acid.

### RELATED APPLICATIONS

This application claims priority from Provisional Application U.S. S. No. 60/379,461, filed May 9, 2002 which is incorporated by reference in its entirety.

### FIELD OF THE INVENTION

This invention relates generally to methods for determining the haplotype of a DNA sample.

### BACKGROUND OF THE INVENTION

Natural sequence variation (i.e., polymorphism) is a fundamental property of all genomic information. Any two human chromosomes (haploids) show multiple sites and types of polymorphisms. Some polymorphisms have functional consequences, and cause or implicate disease, whereas many do not.

Conventional methods of haplotyping require DNA amplification by PCR or cloning and extensive optimization. These methods are slow, labor intensive and expensive to perform, and not suited for large-scale association studies, or routine clinical diagnostics. Current technology involved in the analysis of DNA is also inherently limited in the ability to analyze large populations. Genetic methods typically rely on the individual analysis of regions of the genome. For instance, one PCR reaction is needed per data point. The study of populations would thus require the SNPs to be analyzed independently in a one by one fashion which leads to high cost and long times in analysis. The time and cost analysis of a SNP by current methodologies is between four and five hours (unmultiplexed) and approximately one dollar, regardless of the technology employed. These methods include different methods such as mass spectrometry, bead arrays, cleavase assays, hybridization arrays, primer extension, capillary electrophoresis, and TaqMan. Analysis of 5,000 SNPs, as estimated in an academic laboratory setting of one 96-well real-time PCR machine, would take approximately 10 days and $5,000, with continual running, and this does not include the time for generation of haplotype information.

There is a need in the field for a fast inexpensive method for haplotyping to be used with large sample populations and long stretches of DNA.

### SUMMARY OF THE INVENTION

The method and apparatus of the invention involves direct linear analysis of several kilobase lengths of DNA for the generation of haplotypes. The invention provides for a method of detecting multiple markers on a segment of DNA and determining the distance between these markers. The invention can be used to rapidly haplotype DNA simultaneously at several loci.

The invention is broadly drawn to a method for rapidly haplotyping a nucleic acid, the nucleic acid being DNA or RNA. In one aspect of the invention, a method of determining the haplotype of a subject is described. The method includes providing an extended polynucleotide derived from a subject that includes a plurality of target sites that are each similarly labeled with at least a first unit-specific marker and a second unit-specific marker (the first unit-specific marker and second unit-specific marker provide information for a haplotype in said subject); moving the nucleic acid relative to a stationary detection station, and detecting the plurality of labeled sites at the detection station, thereby determining a haplotype of a subject.

In one embodiment of this embodiment, the target sites are base sequence variations such as single nucleotide polymorphisms, multibase deletions, multibase insertions, microsatellite repeats, dinucleotide repeats, tri-nucleotide repeats, sequence rearrangements, or chimeric sequences.

In another embodiment of this embodiment, the unit specific markers are luminescent hybridization probes that have a distinguishable characteristic. Such distinguishable characteristics include luminescence emission spectral distribution, lifetime, intensity, burst duration, and polarization anisotropy.

In another embodiment of the embodiment, the hybridization probe can be DNA, RNA, locked nucleic acids (LNA), and peptide nucleic acids (PNA). In another embodiment of the embodiment, the luminescent hybridization probes include single dye molecules, energy transfer dye pairs, nano-particles, luminescent nano-crystals, intercalating dyes, molecular beacons and quantum dots. In another embodiment of the invention, each luminescent hybridization probe specifically hybridizes to one of the plurality of target sites.

In another embodiment, the method of the invention further includes a third unit-specific marker that provides information for a haplotype in a subject. In yet another embodiment, the method of the invention further also includes a fourth unit-specific marker, which provides information for a haplotype in the subject.

The subject can be, for example, a mammalian subject, such as a human.

In another embodiment, the unit specific markers are single probes that are specific for each target or multiple probes that act together to identify the target. The single probes of the invention can be oligo DNA, oligo RNA, oligo beacons, oligo peptide nucleic acids, oligo locked nucleic acids, and chimeric oligos. The multiple probes of the invention can be hybridization pairs, invader oligo pairs, ligation oligo pairs, mismatch extension 5′-exonuclease oligo pairs, energy transfer oligo pairs, and 3′-exonuclease pairs.

The invention can be used to analyze any nucleic acid, such as DNA or RNA, including PCR-amplified DNA or PCR-amplified DNA.

In one embodiment of the invention, the stationary detection station is in optical communication with an avalanche photo diode or a charge coupled device.

In another embodiment, the nucleic acid is moved relative to the stationary detection station through the action of at least one molecular motor. In yet another embodiment, the nucleic acid is moved relative to the stationary detection station through the action of a plurality of molecular motors in solution. In yet another embodiment, the nucleic acid is moved relative to the stationary detection station through the action of hydrodynamic force.

In another embodiment, the detection station includes at least one donor fluorophore and the first unit specific marker and a second unit specific marker each include at least one acceptor fluorophore. In another embodiment, the detection station includes at least one acceptor fluorophore and the first unit specific marker and second unit specific marker each include at least one donor fluorophore.

In some aspects, the detection station detects fluorescence resonance energy transfer.

In another embodiment, analysis of the nucleic acid by the method of the invention provides information about the linear arrangement of target sites within the nucleic acid.

In another embodiment, the detection station detects the plurality of target sites of the nucleic acid simultaneously. In one embodiment, the unit specific markers are detected by a confocal microscope. In another embodiment, the plurality of target sites are distinguished by labeling each of said plurality of sites with a different colored luminescent hybridization probe.

In another aspect, the invention provides a method of determining a haplotype of a subject, the method includes moving an extended polynucleotide derived from the subject which includes a plurality of selected genetic markers which are each labeled with at least one distinguishable unit-specific marker, where the plurality of selected genetic markers provides information for a haplotype in said subject, through a channel; exposing the plurality of labeled selected genetic markers to a detection station as the units move relative to the detection station, where the plurality of sites interacts with the detection station to produce a detectable signal within the channel or at the edge of the channel; and detecting sequentially the signals resulting from said interaction to analyze the polynucleotide, thereby determining a haplotype of a subject.

In one embodiment, the detection station includes an agent selected from electromagnetic radiation, a quenching source and a fluorescence excitation source. The agent can include a fluorescence excitation source and the first unit-specific marker and the second unit-specific marker include fluorescent hybridization probes.

In another embodiment, the invention includes a method for determining a haplotype of a population of nucleic acids in a pool of nucleic acids, which includes at least a first population and at least a second population of nucleic acids, where the method includes providing a pool of extended polynucleotides, where the polynucleotides in a population comprise a plurality of target sites that are each similarly labeled with at least a first unit-specific marker and a second unit-specific marker, where the at least first unit-specific marker and second unit-specific marker provide information for a haplotype in the pool, further wherein the target sites are selected genetic markers; moving the polynucleotides of the pool past a stationary detection station; detecting the luminescent hybridization probes at the stationary detection station; and measuring said luminescent probes as the polynucleotides pass by the detectors, thereby determining the haplotype of the species of the polynucleotides in the pool.

In one embodiment, the target sites are base sequence variations selected from single nucleotide polymorphism, multibase deletion, multibase insertion, microsatellite repeats, dinucleotide repeats, tri-nucleotide repeats, sequence rearrangements, and chimeric sequence.

In another embodiment, the unit specific markers are luminescent hybridization probes that have a distinguishable characteristic. The distinguishable characteristic can be, for example, luminescence emission spectral distribution, lifetime, intensity, burst duration, and polarization anisotropy.

The luminescent hybridization probes include, for example, single dye molecules, energy transfer dye pairs, nano-particles, quantum dots, luminescent nano-crystals, intercalating dyes, or molecular beacons.

In some embodiments, each luminescent hybridization probe specifically hybridizes to one of the plurality of target sites. The luminescent hybridization probes can be, for example, DNA, RNA, locked nucleic acids, or peptide nucleic acids.

In additional embodiments, the population of nucleic acids includes a third unit-specific marker that provides information for a haplotype in the pool. In additional embodiments, the population of nucleic acids includes a fourth unit-specific marker that provides information for a haplotype in the pool.

In some embodiments, the unit specific markers are single probes that are specific for each target or multiple probes that act together to identify the target. The single probes can be, for example, oligo DNA, oligo RNA, oligo beacon, oligo peptide nucleic acids, oligo locked nucleic acids, and chimeric oligos. The multiple probes can be, for example, hybridization pairs, invader oligo pairs, ligation oligo pairs, mismatch extension 5′-exonuclease oligo pairs, energy transfer oligo pairs, and 3′-exonuclease pairs.

The polynucleotides in a pool can be, e.g., DNA, RNA, or mixtures thereof.

In some aspects, the stationary detection station is in optical communication with an avalanche photo diode or a charge coupled device.

In some embodiments, the detection station detects fluorescence resonance energy transfer. In some aspects of the invention, the detection station includes at least one donor fluorophore and the first unit specific marker and second unit specific marker each include at least one acceptor fluorophore. In other embodiments, the detection station includes at least one acceptor fluorophore and the first unit specific marker and second unit specific marker each include at least one donor fluorophore.

In another embodiment, the plurality of sites are distinguished by labeling each of the plurality of sites with a different colored luminescent hybridization probe. In one embodiment, the first population includes polynucleotides from one individual and the second population includes polynucleotides from a different individual. In another embodiment, the first population includes polynucleotides from a healthy state of a subject and the second population comprises polynucleotides from a disease state of the same subject. The subject can be, for example, a mammal, such as a human.

In another aspect, the invention includes a method of determining a haplotype of a subject, by providing a polynucleotide, a first ligation oligonucleotide and a second ligation oligonucleotide, where the first ligation oligonucleotide is associated with a first labeled moiety and includes a first constant sequence complementary to a sequence in the target polynucleotide that provides information for a haplotype in said subject, a query nucleotide at the 3′ terminus of said first ligation polynucleotide and, optionally, a mismatch oligonucleotide adjacent to the query nucleotide. The second ligation oligonucleotide is associated with a second labeled moiety and includes a second constant sequence complementary to a sequence in the target polynucleotide that provides information for a haplotype in said subject, a query nucleotide at the 3′ terminus of said second ligation polynucleotide and, optionally, a mismatch oligonucleotide adjacent to the query nucleotide. An effective amount of the first ligation oligonucleotide is annealed to the polynucleotide to yield a primed first template, which is combined with an effective amount of a polymerase enzyme and at least two types of nucleotide triphosphates, under conditions sufficient for polymerase activity, thereby forming a first elongated polynucleotide. An effective amount of the second ligation oligonucleotide is annealed to the polynucleotide to yield a primed second template, which is combined with an effective amount of a polymerase enzyme and at least two types of nucleotide triphosphates, under conditions sufficient for polymerase activity, thereby forming a second elongated polynucleotide. The elongated first polynucleotide and the elongated second polynucleotide are extended; and the first labeled moiety and second labeled moiety are detected, thereby determining a haplotype.

In another aspect the labeled moieties are detected by moving the extended elongated polynucleotides relative to a stationary detection station. In another aspect of the embodiment, the fluorescence resonance energy transfer is detected.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a schematic representation of fragment haplotyping using single molecule analysis. (1) amplification of the target DNA; (2) hybridization of detectably labeled probes complementary to DNA sequences unique to various alleles; (3) detection of labeled DNA which is extended and passed by sensors that detect the labeled moieties and their location on the DNA sequence; (4) readout from the sensors depicting the amount of label detected, and in what position it was detected.

FIG. 2 is a schematic of the use of long PCR and fluorescent bases in haplotyping of single nucleotide polymorphisms (SNPs). The four different haplotypes are labeled with four different color combinations. In this Figure, the different combinations are shown as haplotype A with T (bold) and C (italic); haplotype B with C (italic); haplotype C with T (bold) and haplotype D.

FIG. 3 is a sample output from the use of the haplotyping method for SNPs shown in FIG. 2. Haplotype A may be labeled with color 1 on the thymidine (here shown as T), color 2 on the cytosine (here shown as C), and an intercalant. Haplotype B is labeled with color 2 on the cytosine (here shown as C) and an intercalant. Haplotype C is labeled with color 1 on the thymidine (here shown as T) and an intercalant. Haplotype D is labeled with an intercalant.

FIG. 4 is a schematic showing a primer extension four color analysis method for haplotyping SNPs. In this schematic T is labeled with a first color, e.g. orange (IR-represented by a long dash followed by a short dash), C is labeled with a second color, e.g. red (R-represented by a long dashed line), A is labeled with a third color, e.g. green (G-represented by a solid line), and the DNA is labeled with an intercalator labeled a fourth color, e.g. blue (B). The assay is set up as the three color analysis presented in FIG. 3.

FIG. 5 is a schematic representing the confirmation of haplotyping results on one DNA strand by assaying the complementary strand using the same mixture of fluorescently labeled dNTPs.

FIG. 6 is a schematic representing the label combinations that would be present in a three SNP analysis. C is color 1 (represented by a long dashed line), G is color 2 (represented by a short dashed line), T is color 3 (represented by a long dash followed by a short dash), and A is color 4 (represented by a solid line).

FIG. 7 is a schematic representing a more effective labeling scheme for haplotyping three SNPs when compared to the scheme in FIG. 6. One possible labeling scheme is the A, T and G are labeled with a different fluorophore and the biallelic nature of SNPs allows the matching of the 8 different possible haplotypes with a unique color scheme.

FIG. 8 is a schematic representing a labeling scheme for a five SNP haplotyping assay. The first four SNPs, A, C, T and G are labeled with four different colors. The fifth G is distinguished through the use of mixture tagging approach where the dGTP is represented by a 50/50 population of dGTP labeled with color 4 (represented by a short dashed line) and dGTP labeled with color 5 (represented by a long dash with two short dashes).

FIG. 9 is a schematic of a confocal optical system that allows four color analysis to be performed.

FIG. 10 is a schematic showing the steps used to analyze haplotypes of subjects with a certain phenotype, and graphs showing the presence of certain SNPs in the two populations.

FIG. 11 is a schematic showing two methods for analyzing the pooled haplotypes of a population. In the upper schematic, pooled DNA undergoes long PCR, followed by denaturation into ssDNA. In this type of analysis the different probes do not have to be differentially labeled because it is their position on the DNA that is measured to identify the haplotype.

In the lower schematic the pooled DNA is labeled with fluorescent primers, each specific for a particular SNP. These primers are then detected through direct fluorescent readout using single molecule analysis.

FIG. 12 is a schematic showing, at the top, the sequences of two alleles of two SNPs: A, a, B, and b. At the bottom, the schematic shows a method of annealing an oligo to the DNA in question (shown at top).

FIG. 13 is a schematic showing differential labeling of PCR products for DNAs with different SNPs at position A. Differential labeling of the same oligo can be used to confirm results. In the situation on the left, oligos labeled with IRD800 are specific for the “b” SNP at one locus, oligos labeled with TAMRA are specific for the “A” SNP, and oligos labeled with Cy5 are specific for the “a” SNP or vice versa. In the situation on the right, oligos labeled with CyS are specific for the “b” SNP at one locus, oligos labeled with TAMRA are specific for the “A” SNP, and oligos labeled with IRD800 are specific for the “a” SNP or vice versa.

FIG. 14 is a schematic showing that the same is true for the B locus, where the oligos used to detect the “B” and “b” SNPs are differentially labeled with TAMRA.

FIG. 15 is a gel photograph showing TAMRA fluorescence without staining (above) and ethidium bromide staining (below). In lanes 1 and 4, the SNP present is labeled with TAMRA. The gels (below) show that PCR product was produced in all cases in which the template was present, it was just not labeled with TAMRA.

FIG. 16 is a gel photograph showing TAMRA fluorescence associated with the B locus.

FIG. 17 is a gel photograph densitometrically measured for TAMRA fluorescence relative to ethidium bromide fluorescence.

FIG. 18 is a series of graphs showing fluorescence densitometry under various labeling and different SNP combinations.

FIG. 19 are two graphs showing the results of correlation analysis of the previous fluorescence densitometry.

### DETAILED DESCRIPTION OF THE INVENTION

As used herein, the terms “selective,” “selectively,” “specific,” “specifically,” “essentially,” “uniformly” and the like, mean that the indicated event occurs to a particular degree. In particular, the percent identity of a nucleotide to its hybridization target is greater than 90%, preferably greater than 95%, most preferably, greater than 99%.

As used herein the term “unit specific information” refers to any structural information about one, some, or all of the units of a nucleic acid polymer. The structural information obtained by analyzing a nucleic acid according to the methods of the invention may include the identification of characteristic properties of the nucleic acid which (in turn) allows, for example, for the identification of the presence of a nucleic acid in a sample or a determination of the relatedness of nucleic acid polymers, identification of the size of the nucleic acid polymer, identification of the proximity or distance between two or more individual units or unit specific markers in a nucleic acid polymer, identification of the order of two or more individual units or unit specific markers within a nucleic acid polymer, and/or identification of the general composition of the units or unit specific markers of the nucleic acid polymer. Since the structure and function of biological molecules are interdependent, the structural information can reveal important information about the function of the nucleic acid polymer. The invention is a method for analyzing nucleic acid polymers based on a compilation of data obtained from incomplete labeling of the nucleic acid polymers. The methods can be performed using data generated from single unit labels or multiple unit labels (both referred to herein as unit specific markers), single stranded nucleic acid polymers, double stranded nucleic acid polymers, or combinations thereof.

As used herein, the term “target site” refers to sequences on a nucleic acid where the sequence varies. Examples include, but are not limited to, polymorphisms which exist in different forms such as single nucleotide variations, nucleotide repeats, multibase deletion (more than one nucleotide deleted from the consensus sequence), multibase insertion (more than one nucleotide inserted from the consensus sequence), microsatellite repeats (small numbers of nucleotide repeats with a typical 5-1000 repeat units), di-nucleotide repeats, tri-nucleotide repeats, sequence rearrangements (including translocation and duplication), chimeric sequence (two sequences from different gene origins are fused together), and the like. Among sequence polymorphisms, the most frequent polymorphisms in the human genome are single-base variations, also called single-nucleotide polymorphisms (SNPs). SNPs are abundant, stable and widely distributed across the genome.

Unit specific markers are used herein as probes which bind, anneal or hybridize to a specific target site. As used herein, the term “probe” refers to a substance which binds, anneals or hybridizes to a specific target site. Examples of probes include, but are not limited to, DNA, RNA, locked nucleic acids (LNA), peptide nucleic acids (PNA), beacon oligonucleotide (oligo), or chimeric oligo. The unit specific marker can be, for example, a series of distinct nucleic acid probes selected from two base pair probes, three base pair probes, four base pair probes, and five base pair probes.

PNA is a nucleic acid analog where the sugar phosphate backbone has been replaced with a peptide backbone generally composed of 2-aminoethyl-glycine linkages. Nucleic acid bases are connected to the backbone through methyl carbonyl linkers to the amino nitrogens. The resulting analog is uncharged and achiral while maintaining its ability to recognize DNA and RNA through Watson-Crick base pairing. PNAs are resistant to enzymatic degradation and are stable in living cells. Generally, hybrids between PNA and nucleic acids display enhanced thermodynamic stability and unique ionic characteristics.

LNA, locked nucleic acid, is an RNA-derivative used in the synthesis of RNA oligomers. LNA, unlike PNA, has the same phosphate backbone found in DNA and RNA which allows LNA oligomers to be formed by the formation of a phosphodiester bond. LNA differs from RNA in that the nucleotides contain a methylene bridge that links the 2′-oxygen of the ribose with the 4′-carbon. This results in a locked 3′-endo conformation in the sugar that reduces the conformational flexibility of the ribose and increases the organization of the phosphate backbone. This modification increases the binding affinity of LNAs to their complimentary nucleic acid.

A “labeled unit specific marker” as used herein is any unit specific marker in a polymer that identifies a particular unit or units. A labeled unit specific marker includes, for instance, fluorescent markers which are bound to a particular unit or units, proteins, peptides, nucleic acids, polysaccharides, short oligomers, tRNA, etc. that recognize and bind to a particular unit or units and that can be detected by e.g., possessing an intrinsically labeled property or including an extrinsic label or by binding to another detection molecule such as an antibody.

A labeled unit specific marker as used herein is labeled so as to have a “distinguishable characteristic” when its label is distinct from at least one other labeled unit specific marker. Typically, labeled unit specific markers will be distinguished from each other based on distinct luminescence emission spectral distribution, lifetime, intensity, burst duration, or polarization anisotropy.

The nucleic acid analysis described herein can be used to identify DNA fragments by analyzing the hybridization patterns of multiple probes to individual fragments of polymers. The number, type, order, and distance between the multiple probes bound to an unknown fragment of DNA can be determined. This information can be used to identify the number of differentially expressed genes unambiguously. Furthermore, the methods of the invention are able to quantitate precisely the actual number of particular expressed genes. Given the great amount of information generated, the methods of the invention do not require a selection of expressed genes or unknown nucleic acids to be assayed. The methods of the invention can identify the unknown expressed genes by computer analysis of the hybridization patterns generated. The data obtained from linear analysis of the DNA probes are then matched with information in a database to determine the composition or identity of the target DNA. The methods can thus be used to determine haplotypes and to analyze information from hybridization reactions, which can then be applied to diagnostics and determination of gene expression patterns.

As used herein, the term “single probe” refers to a luminescent hybridization probe that anneals or hybridizes specifically to a target sequence.

As used herein, the term, “multiple probes,” refers to luminescent hybridization probes that act together to identify the target site. Multiple probes can act together by annealing or hybridizing specifically to the same target site, annealing or hybridizing specifically to multiple target sites, using probes with different binding affinities so that specific annealing or hybridization to the target site occurs at a particular temperature. The multiple probes can be hybridization pairs, 5′-exonuclease oligonucleotide pairs, energy transfer oligonucleotide pairs, or 3′-exonuclease pairs.

A labeled moiety is a moiety which is detectable. Examples of labeled moieties include dyes (e.g., fluorescent dye molecules), quantum dots, a luminescent nano-crystal, molecular beacons and radioactive particles.

As used herein, the term “luminescent hybridization probe” refers to probes which have distinguishable characteristics, including luminescence emission, which can be differentiated by spectral distribution, lifetime distribution, intensity distribution, or polarization anisotropy distribution of the luminescence. These distributions are caused by the luminescent hybridization probe also having a single dye molecule, an energy transfer pair, a nano-particle, a quantum dot, a luminescent nano-crystal, or a molecular beacon.

The term, “molecular beacon,” refers to a system that reports the presence of specific nucleic acids in homogeneous solution. These probes undergo a spontaneous fluorogenic conformational change when they hybridize to their target. Only perfectly complementary targets elicit this response, as specific annealing or hybridization does not occur when the target contains a mismatched nucleotide or a deletion.

Fluorescence resonance energy transfer (FRET) is a distance-dependent interaction between the electronic excited states of two dye molecules in which excitation is transferred from a donor molecule to an acceptor molecule without emission of a photon. FRET is dependent on the inverse sixth power of the intermolecular separation, making it useful over distances comparable with the dimensions of biological macromolecules. Thus, FRET is an important technique for investigating a variety of biological phenomena that produce changes in molecular proximity.

Donor and acceptor molecules must be in close proximity (typically 10-100 Å). Absorption spectrum of the acceptor must overlap fluorescence emission spectrum of the donor. In most applications, the donor and acceptor dyes are different, in which case FRET can be detected by the appearance of sensitized fluorescence of the acceptor or by quenching of donor fluorescence. When the donor and acceptor are the same, FRET can be detected by the resulting fluorescence depolarization.

On excitation, the donor fluorophore emits fluorescence photons with a characteristic lifetime (t). The close proximity (5 to 10 nm) of a second fluorophore with an absorption band which overlaps with the emission band of the donor leads to its excitation (acceptor) at a rate which is inversely proportional to the sixth power of the distance between them. The donor fluorescence and its lifetime are therefore dependent on donor-acceptor distance. Measurements of FRET can be based on the changes in fluorescence intensity or on the measurement of the donor fluorescence lifetime.

The technique makes use of some unusual properties of dye molecules. In FRET measurements that use fluorescent dyes, the dye molecule is typically excited at one wavelength of light and data is collected at a longer wavelength.

As used herein, “energy transfer dye pair” refers to a distance dependent interaction between the electronic excited states of two dye molecules in which excitation is transferred from one dye (the donor fluorophore) to another dye (the acceptor fluorophore) without emission of a photon. The two dye molecules are generally located on opposite sides of a cleavable modified nucleotide such that cleavage will alter the proximity of the dyes to one another and thereby change the fluorescence output of the dyes on the polynucleotide.

As used herein, a “quantum dot,” is a label used with a luminescent hybridization probe which comprises a core, a cap and a hydrophilic attachment group. The “core” is a nanoparticle-sized semiconductor. The semiconductor ranges in size from about 1 nm to about 10 nm. The core is more preferably a semiconductor and ranges in size from about 2 nm to about 5 nm. Most preferably, the core is CdS or CdSe. In this regard, CdSe is especially preferred as the core, in particular at a size of about 4.2 nm.

The “cap” is a semiconductor that differs from the semiconductor of the core and binds to the core, thereby forming a surface layer on the core. The cap must be such that, upon combination with a given semiconductor core, results in a luminescent quantum dot. Preferably, the cap is ZnS or CdS. More preferably, the cap is ZnS. In particular, the cap is preferably ZnS when the core is CdSe or CdS and the cap is preferably CdS when the core is CdSe.

The “attachment group” as that term is used herein, refers to any organic group that can be attached, such as by any stable physical or chemical association, to the surface of the cap of the luminescent semiconductor quantum dot and can render the quantum dot water-soluble without rendering the quantum dot no longer luminescent. Accordingly, the attachment group comprises a hydrophilic moiety. Preferably, the attachment group is mercaptoacetic acid.

As used herein, a “fluorescent nano-crystal,” is a crystal used on a luminescent hybridization probe that resists photobleaching, shares an excitation wavelength spectrum, and is capable of emitting fluorescence of high quantum yield and with discrete peak emission spectra.

As used herein, a “fluorescent nano-particle,” is a particle used on a luminescent hybridization probe that resists photobleaching, shares an excitation wavelength spectrum, and is capable of emitting fluorescence of high quantum yield and with discrete peak emission spectra.

As used herein, a “single dye,” is a fluorescent label used with an luminescent hybridization probe. This label can include TAMRA, Cy3, Cy5, Cascade Blue, and IR800.

As used herein, an “intercalating dye,” is a dye that is capable of labeling nucleic acid by interacting hydrophobically with the nucleic acid. An example of an intercalating dye is ethidium bromide.

As used herein, the term “invader oligonucleotide” refers to an oligonucleotide which contains sequences at its 3′ end which are substantially the same as sequences located at the 5′ end of a probe oligonucleotide; these regions will compete for hybridization to the target site along a complementary target nucleic acid.

As used herein, the term “ligation oligonucleotide”, refers to an oligonucleotide which is complementary to at least a portion of the target site and which is hybridized to the target site to form a hybrid having a single stranded region and a double stranded region. The hybrid can be contacted with a plurality of oligonucleotide triphosphates (e.g., ATP, CTP, GTP, TTP, UTP) and a polymerase enzyme so as to ligate to the hybrid, in sequence, at least some of the plurality of oligonucleotide triphosphates to extend the double stranded region and thereby synthesize a nucleic acid strand which is complementary to the portion of the target site.

As used herein, a “query nucleotide” is a nucleotide at the 3′ terminus of the ligation oligonucleotide. The query nucleotide can form a base pair or a mismatch with the target site to which the ligation oligonucleotide is hybridized.

As used herein, a “mismatch extension exonuclease oligonucleotide,” is an oligonucleotide that relies on the measurement of the difference in primer extension efficiency by a DNA polymerase of a matched over a mismatched 5′ or 3′ terminal. In one example two detection primers differing with one base at the 3′-end are designed; one precisely complementary to one specie of the target site DNA-sequence and the other precisely complementary another specie of the target site DNA-sequence. The primers are hybridized with the 3′-termini over the base of interest and the primer extension rates are, after incubation with DNA polymerase and deoxynucleotides, measured. If the detection primer exactly matches to the template a high extension rate will be observed. In contrast, if the 3′-end of the detection primer does not exactly match to the template (mismatch) the primer extension rate will be much lower. The difference in primer extension efficiency by the DNA polymerase of a matched over a mismatched 3′-terminal can then be used for single-base discrimination.

As used herein, a “sample chamber” is a container where the sample is prepared for interrogation. Sample chambers include, for example, a flow-through capillary, glass slide with coverslip, microchip, or any other suitable sample chamber that allows handling of the sample. A sample chamber can be, for example, a channel through which the sample flows.

A sample can flow through a channel by a variety of different means, such as by a molecular motor, by electrophoretic force, by hydrodynamic force, or combinations thereof.

A “detection station” as used herein is a region in a sample chamber where a marker on a sample is interrogated. The interrogation is accomplished be detecting signals emitted from the marker itself (intrinsic) or by contacting the marker with an agent which causes it to generate a detectable signal. The detection station may be composed of any material including a gas. Preferably the station is a non-liquid material. “Non-liquid” has its ordinary meaning in the art. A liquid is a non-solid, non-gaseous material characterized by free movement of its constituent molecules among themselves but without the tendency to separate. In another preferred embodiment the station is a solid material. A detection station may be associated with a device which enables detection of a sample which is interrogated at the detection station. For example, the detection station may be in optical communication with an avalanche photo diode or a charge coupled device. A detection station can be, for example, a confocal laser spot which is focused in a channel through which a labeled sample flows. The laser can be remote and directed to the location through an optical train.

When an interaction between a unit specific marker and the detection station produces a polymer-dependent impulse, the station is a “signal generation station”. One type of signal generation station is an interaction station. As used herein, an “interaction station or site” is a region where a unit specific marker of the polymer interacts with an agent and is positioned with respect to the agent in close enough proximity whereby they can interact. The interaction station for fluorophores, for example, is that region where they are close enough so that they energetically interact to produce a signal.

As used herein, an “extended polynucleotide” or an “extended nucleic acid” is a nucleic acid which is not coiled or supercoiled, e.g., it is stretched so that is approximately linear.

The invention is broadly related to the use of single molecule genetic analysis to analyze large populations of nucleic acids. In one embodiment of the invention, direct analysis of DNA allows for pooling of DNA to look at populations and determine whether or not the populations have differences at particular locations in the genome. This allows the DNA from the reactions to be pooled, prepared together in the same reaction tube, and then analyzed as separate single molecules which are indicative of the states present in a particular population. The states of each of the test sample population and control population are compared to identify differences between the populations and, hence, to address the question of whether there is a genetic difference between the populations. This is particularly useful when populations are separated by phenotypic differences such as disease and non-disease.

In another embodiment of the invention, the DNA from a population of case and control populations are separately pooled into their respective single reaction vessels. Each reaction tube thus contains the equivalent amount of DNA to all the DNA of one subset of the population, either the test sample or control population. A selected locus of the population is chosen for analysis using long PCR. The long PCR reaction is performed on the different reaction mixtures, thus amplifying all the respective haplotypes present in each of the reaction mixtures. The resulting PCR product mixture from each of the populations is then tagged, cleaned, and sent through the single molecule detection system. Comparison of the test sample and control reaction mixtures thus allows for the determination of particular haplotypes present or absent in each of the respective populations. Other embodiments of the system relate to different methods of sample preparation including methods that do not involve the amplification of the regions of interest, but instead, involve direct tagging of the sites of the DNA using sequence-specific tags. Additional embodiments also involve targeted cloning methods for the recognition of particular regions of the genome to be analyzed.

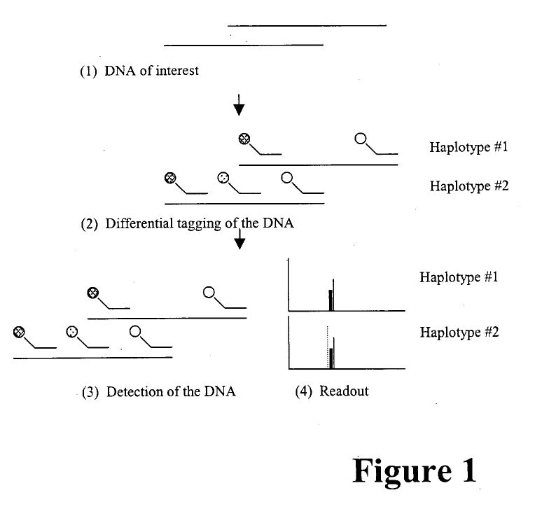

The invention is also broadly drawn to fragment haplotyping that can be performed through the use of single molecule analysis. In one embodiment of the invention, the method involves the use of multiplexed single molecule detection. For instance, haplotype analysis over a region of the genome may involve isolation/amplification of the DNA fragment, differential tagging of the polymorphic regions of the genome, and discrimination of the differential tagging patterns using single molecule detection. This method is described schematically in FIG. 1.

In a specific embodiment, the invention is drawn to a method for detecting single-nucleotide polymorphisms at two distinct loci. The method relies on the amplification of DNA using fluorescently-labeled oligonucleotides that can discriminate between templates differing in sequence at only one position. The simultaneous use of multiple discriminatory oligos, each conjugated with different fluorophores, allows simultaneous amplification of multiple loci. The PCR products are analyzed on an analysis platform such as the GENEENGINE™ which is adapted for analysis of single molecules and molecular populations. Details of one such system are found in U.S. Pat. No. 6,355,420, which is specifically incorporated by reference herein. Single-molecule detection mode is used to test whether a set of fluorophores is correlated, which indicates the linkage of alleles, and hence, identification of a haplotype.

Haplotyping

SNPs serve as ideal genetic markers for complex disease association studies (linkage analysis), in which particular alleles (one of two or more genes that occur at the same locus of homologous chromosomes) of genetic markers (haplotype) in close proximity to a disease mutation are consistently associated with the disease. Comparison of newly deduced SNP information to a known SNP library allows the elucidation of complex genetic components of human disease, thereby accelerating the drug discovery process. SNPs are also good markers for population, evolution and forensic studies, and polymorphism profiles. They offer the potential to assess a disease risk or predict a drug response based on an individual's genetic profile. Further, SNP profiles may be used to tailor drug treatments to individual patients, to improve the efficacy and safety of the treatment.

The haplotype is a set of genetic determinants located on a single chromosome and it typically contains a particular combination of alleles (all the alternative sequences of a gene) in a region of a chromosome. In other words, the haplotype is phased sequence information on individual chromosomes. Very often, phased SNPs on a chromosome defines a haplotype. The combination of two haplotypes on two human chromosomes ultimately determines the genetic profile of a human cell. It is the haplotype that determines a linkage between a specific genetic marker and a disease mutation. Current methods of scoring SNPs, such as hybridization microarray or direct gel sequencing, (reviewed in Landgren et al., Genome Research, 8:769-776, 1998) can accurately type individual SNPs, but can not determine which chromosome of a diploid pair is associated with each polymorphism. Without the phased information of haplotypes, it may be impossible to detect the diploid association due to the numerous possibilities of different haplotypes.

The haplotype deduced from a genotype is typically done in bulk as follows (inferred haplotypes): the region of interest is PCR amplified, the genotype is determined, and the haplotype is deduced from homozygous individuals. Since the genotype is based on a bulk measurement on a mixture of both chromosomes, this genotyping approach has serious limitations for large numbers of SNP markers.

Current genomics technologies used for the analysis of populations include methods based on Sanger sequencing and PCR or other types of enzymatic amplification. Because haplotypes need to be generated over several kilobases of DNA to megabase sizes of DNA for analysis of inheritance, both PCR and Sanger sequencing dependent technologies are often insufficient. Thus, existing methods of genetic analysis do not allow the identification of haplotypes over long stretches of DNA.

Traditionally, association studies have been successful only for simple, monogenic diseases involving a small number of markers, where the possible combinations of different haplotypes are limited. Therefore, the haplotypes can be typically deduced from genotypes by typing many individuals and by the availability of homozygotes and parental information. However, most diseases are complex and involve multiple genes. For polygenic association studies, many more markers are needed and, therefore, the number of possible haplotypes is large. In these cases, it is extremely difficult to infer the haplotype from the genotype.

One method of haplotyping uses microsatellite markers at certain positions in the genome (for example 100 kb, 200 kb, and 300 kb). Assaying position 100 kb with PCR yields two different sized PCR products, one denoted “A” and another denoted “a”. Positions 200 kb and 300 kb likewise yields Bb and Cc. In order to understand inheritance, it is important to know which microsatellite markers are co-inherited, allowing one to determine which combination of markers give rise to a particular phenotypic trait. There are many possible combinations of the markers on the two copies of the locus. The goal is to determine the haplotype, or linear order of physically inherited markers, so that statistical correlation of combinations of markers can be traced to inheritance patterns. With PCR-dependent technologies, analysis of one individual in isolation cannot yield haplotypes. This limitation can be bypassed, however, with the further effort of analyzing the markers of the parents of the individual. With this information, the haplotypes can be statistically reconstructed.

Another method of haplotyping uses single nucleotide polymorphisms (SNPs) to determine ancestral (not familial) inheritance. Many SNPs are scored in order to find shared regions of DNA that have been passed down from one common ancestor. These shared regions of the genome, stipulated to be around 30 kilobases, can then be linked to phenotypic traits of interest. In order to determine which regions are the regions that have been shuffled, haplotype determination is critical. However, SNP analysis of candidate gene regions is difficult with current technologies, parental information is required to assemble phenotypes and, because SNPs are biallelic (having only two possible polymorphisms), stretches of several heterozygous biallelic SNPs cannot be assembled into haplotypes using current technology. As a result, this information is lost and cannot be recovered, even with complete lineages assayed.

Another conventional alternative for haplotyping is allele-specific polymerase chain reaction (allele-specific PCR, Ruano and Kidd, Nucleic Acids Research, 17:8392, 1989), which is the most commonly used method for direct haplotyping. In these reactions, SNP-specific PCR primers are designed to distinguish and amplify a specific haplotype from two chromosomes. Such reactions require stringent reaction conditions and individual optimization for each target. Therefore, this approach is not suitable for a large scale and high throughput haplotyping. More importantly, such assays are subject to the length limitations of PCR amplification and are not capable of typing SNPs that are more than several kilobases (kb) apart. In addition, such an amplification-based typing is often complicated by the contamination of a small amount of genomic DNA other than the sample DNA during sample handling process.

Other haplotyping methods include single sperm or single chromosome measurements (see, e.g., Ruano et al., PNAS, 87:6296-6300, 1990 and Zhang et al., PNAS, 89:5847-5851, 1992). In a single sperm sorting assay, PCR amplified DNA from individual sorted sperm cells is genotyped. Multiple sperm cells (at least 3-5) from an individual are typed in order to have enough statistical confidence to reveal the two haplotypes. In principle, this sorting approach could be applied to chromosomes. However, this technique is complicated, and, so far, has been successful in only a few research labs.

The molecular cloning method clones a target region of an individual's DNA (or cDNA) into a vector, and genotypes the DNA obtained from single colonies. For each individual, multiple colonies are needed to obtain two haplotypes. This method has been used by many laboratories, but is very labor-intensive, time-consuming and can be difficult to perform in some cases. Researchers are forced to use it because there are no easy alternatives.

Haplotyping by AFM (Atomic Force Microscopy) imaging (Woolley et al., Nature Biotechnology, 18:760-763, 2000 and Taton et al., Nature Biotechnology, 18:713, 2000) allows one to directly visualize the polymorphic sites on individual DNA molecules. This method utilizes AFM with high resolution single walled carbon nanotube probes to read directly multiple polymorphic sites in DNA fragments containing from 100-10,000 bases. This approach involves specific hybridization of labeled oligonucleotide probes to target sequences in DNA fragments followed by direct reading of the presence and spatial localization of the labels by AFM. The throughput and sensitivity of such systems remain to be demonstrated.

Sample Preparation

Locus-specific haplotype analysis requires the isolation of a region of DNA from the genome. These sample preparation methods may involve cloning, PCR, or other methods of DNA isolation. In this embodiment of the invention, the polymerase chain reaction (PCR) is the preferred method of sample isolation. PCR is performed on the region of interest to isolate a region of the genome that is specific for the analysis. PCR can be utilized over a range of several hundred bases to greater than ten-thousand base pairs. The longer the length of the PCR, the greater information of the resultant haplotype determination. Long PCR or other similar technologies can be used to create the desired fragments. PCR is convenient to use in single molecule haplotype analysis because single molecule analysis allows haplotype determination where conventional SNP analysis does not. Cloning or other fragment isolation technologies would allow for conventional SNP analysis because the two copies of a diploid genome are physically separated. In PCR analysis, the two copies of the diploid genome are both amplified, creating a resultant mixture of the two copies of the region of interest. Thus, PCR amplified DNA regions are uniquely suited for analysis using techniques of single molecule detection.

Tagging Chemistry

There are multiple methods of tagging the DNA at the different sites of the genome. These include direct hybridization to the region of interest using sequence-specific oligonucleotides or other hybridizing agents such as peptide nucleic acids. Other methods of tagging include the use of primer extension reactions, ligase-directed sequence detection, and the use of fluorescence resonance energy transfer for the detection of the sequences.

A four nucleotide labeling scheme can be created where the A's, C's, G's, and T's of a target DNA are labeled with different labels. Such a molecule, if moved linearly past a station, will generate a linear order of signals which correspond to the linear sequence of nucleotides on the target DNA. The advantage of using a four nucleotide strategy is its ease of data interpretation and the fact that the entire sequence of unit specific markers can be determined from a single labeled polymer. Adding extrinsic labels to all four bases however, may cause steric hindrance problems. In order to reduce this problem, the intrinsic properties of some or all of the nucleotides may be used to label the nucleotides. As discussed above, nucleotides are intrinsically labeled because each of the purines and pyrimidines have distinct absorption spectra properties. In each of the labeling schemes described herein, the nucleotides may be either extrinsically or intrinsically labeled but it is preferred that at least some of the nucleotides are intrinsically labeled when the four nucleotide labeling method is used. It is also preferred that when extrinsic labels are used with the four nucleotide labeling scheme that the labels be small and neutral in charge to reduce steric hindrance.

A three nucleotide labeling scheme in which three of the four nucleotides are labeled may also be performed. When only three of the four nucleotides are labeled analysis of the data generated by the methods of the invention is more complicated than when all four nucleotides are labeled. The data is more complicated because the number and position of the nucleotides of the fourth unlabeled type must be determined separately. One method for determining the number and position of the fourth nucleotide utilizes analysis of two different sets of labeled nucleic acid molecules. For instance, one nucleic acid molecule may be labeled with A, C, and G, and another with C, G, and T. Analysis of the linear order of labeled nucleotides from the two sets yields sequence data. The three nucleotides chosen for each set can have many different possibilities as long as the two sets contain all four labeled nucleotides. For example, the set ACG can be paired with a set of labeled CGT, ACT or AGT.

The sequence including the fourth nucleotide also may be determined by using only a single labeled polymer rather then a set of at least two differently labeled polymers using a negative labeling strategy to identify the position of the fourth nucleotide on the polymer. Negative labeling involves the identification of sequence information based on units which are not labeled. For instance, when three of the nucleotides of a nucleic acid molecule are labeled with a label which provides a single type of signal, the points along the polymer backbone which are not labeled must be due to the fourth nucleotide. This can be accomplished by determining the distance between labeled nucleotides on a nucleic acid molecule.

dNTPs on oligonucleotides can be labeled with various detectable moieties so that DNA tagged with a labeled oligonucleotide can be detected. dNTPs can be labeled fluorescently with TAMRA, Cy5, Cy3, IRD800, fluoroscein, Texas Red, green fluorescent protein, and other fluorescent labels. dNTPs can be labeled with different colored fluorescers including red, orange, green, purple, and blue. dNTPs can also be labeled radioactively with ³H, ¹⁴C, ³²P, or 125I. DNTPs can also be labeled enzymatically with horseradish peroxidase, alkaline phosphatase, and other enzymatically detectable catalysts. dNTPs can also be labeled by their affinity to other molecules, e.g. avidin-biotin, protein A-IgG, and through other specific interactions.

Primer Extension Mediated Chemistry and Readout

Using primer extension technology, coupled with multi-color analysis of the long PCR-generated fragments, single molecule haplotype analysis can be generated. A DNA sequence to be haplotyped is hybridized with one or more SNP-specific primers. These primers are specific to the sequence just 5′ of the actual SNP. Then one, two or, three of the four nucleotides are labeled with distinguishable labels. (For instance, using dCTP—Cy5 (Amersham) and dUTP—TAMRA (Molecular Probes) fluorescent nucleotides, the haplotypes on a strand of DNA can be determined.) Primer extension is performed in the presence of only the labeled nucleotides. Nucleotides that anneal specifically to the SNP allow the primer to be extended with the labeled nucleotide.

Care must be taken in choosing which SNP and which labeled nucleotide will be analyzed at the same time. If the nucleotide on the 3′ end of the SNP hybridizes to a different labeled nucleotide than the one that hybridized to the SNP itself, it will be double labeled, potentially obscuring the result. Also, if the nucleotide on the 3′ end of the SNP is the same nucleotide as the SNP itself, it can also become labeled with the same nucleotide as the SNP. This can occur for as long as the next nucleotide is the same nucleotide. Care must be taken that that 3′ of this stretch, the next nucleotide does not hybridize with another labeled nucleotide, or the same double labeling will occur as described above. This limitation of the technique can be overcome by not using an excess of labeled nucleotide, so that it is mostly used up during the extension of the first nucleotide.

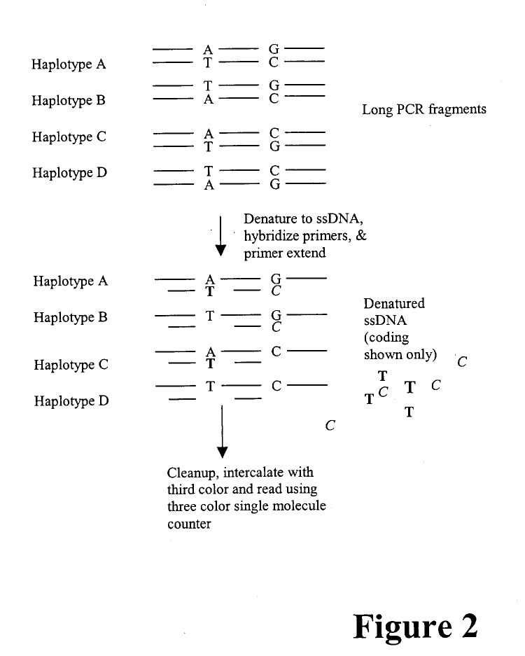

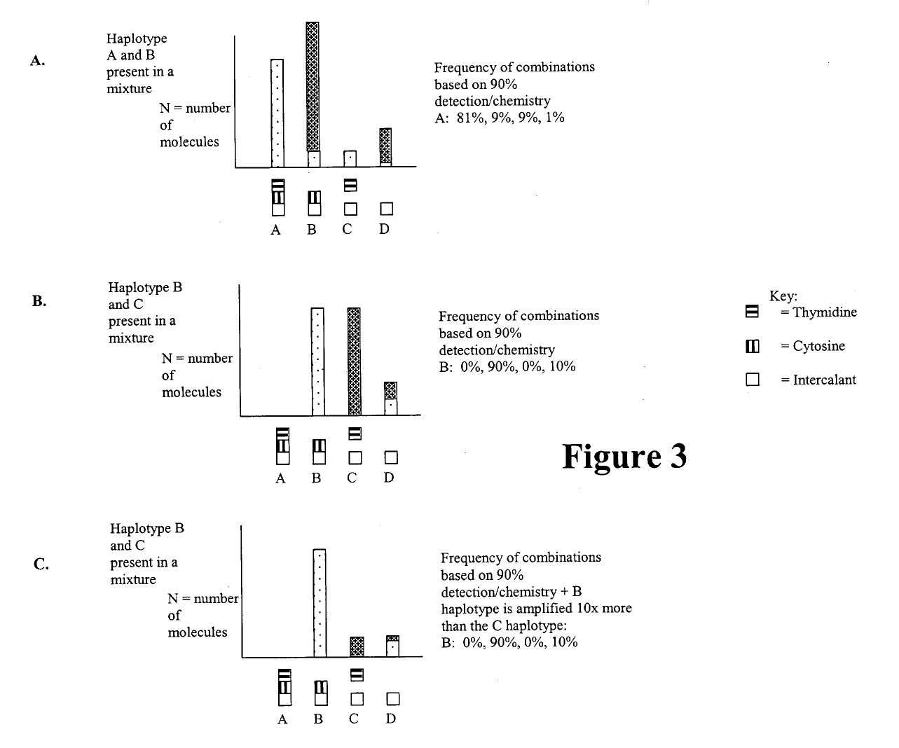

The long PCR fragments are hybridized with a SNP-specific primer and extended in the presence of the two labeled dNTPs only (FIG. 2). Incorporation of the dNTPs only occurs when there is complementarity between the bases. Assay of the two SNPs allows for four different possible haplotypes, 22 versions. Using two different colored dNTPs for the primer extension and a third color (e.g. green) for intercalator of the molecules, four different color combinations allowed for the distinguishing of the different haplotypes. Sample outputs of the data are shown in FIG. 3.

In the sample output in FIG. 3A, both haplotype A and B are present in the mixture. It is assumed that both haplotypes are amplified 50/50 and also that there is 90% detection/chemistry efficiency. The imperfect detection/chemistry creates “background” in the other haplotype channels. For instance, the background arising from the recognition of haplotype A is 9% in haplotype B & C channels and 1% in haplotype D channel. This inefficiency needs to be accounted for in this type of analysis and also could be further mitigated through the use of additional colors for recognition of the sequence sites.

In Panel A, haplotypes A and B are present in equal amounts. When haplotype A is present, assuming 90% detection/chemistry efficiency, one would expect the readout represented by the light dotted bars. There would be 90% signal in the haplotype A channel, 9% in the haplotype B channel, 9% in the haplotype C channel, and 1% in the haplotype D channel. This is due to the greater similarity of haplotypes B and C to haplotype A, than haplotype D to haplotype A. For haplotype A to be mistaken for haplotypes B or C, one fluorescent label would have to be missing or unread, while for haplotype A to be mistaken for haplotype D, two fluorescent labels would have to be missing or unread. When haplotype B is present, again assuming 90% detection/chemistry efficiency, one would expect the readout represented by the dark cross-hatched bars. Haplotype B is labeled with color 2 on the cytosine and an intercalant. There is nearly a 100% chance that the DNA would be labeled with the intercalant, so it does not figure into the calculation of efficiency. Therefore, haplotype B can only be mistaken for haplotype D, if its fluorescent color is missing or unread.

The data analysis requires one to take into account the inefficiencies of detection and labeling chemistry. Furthermore, there may be differential amplification of the two haplotypes present in the mixture (even though they are present in a 1:1 ratio). The following examples illustrate the proposed signal output taking into account both differential amplification of the alleles as well as imperfect chemistry/detection.

In FIG. 3B, it is clear that haplotype B and haplotype C are present in the mixture because haplotype B and C cannot be derived from each other because of inefficiencies, i.e. they are unique in themselves. In Panel B, haplotypes B and C are present in equal amounts. In this case, haplotype B is represented by the light dotted bars in the same proportions as they were in FIG. 3A. The readout for haplotype C is represented by the dark cross-hatched bars. Like haplotype B, haplotype C is labeled with only one fluorophore, and the green intercalant. Haplotype C differs from B in that the fluorophore is color 1 on the thymidine instead of color 2 on the cytosine. Assuming 90% detection/chemistry efficiency, 90% of the readout for haplotype C would show up in the C channel, and 10% in the D channel. Similarly to haplotype D, C had only one fluorophore to lose, so it can only be confused with haplotype D which has no fluorophores attached. Even though the signal indicating haplotype C and haplotype D are at equal values, one must conclude that only haplotypes B and C are in the sample assuming only two haplotypes are in the mixture. This analysis can be extended to other examples and holds true that despite these inefficiencies, haplotypes can still be unambiguously determined with the use of only two differently colored dNTPs and an intercalator color.

In FIG. 3C, again both haplotypes B and C are present, but haplotype B is present at 10 times the amount haplotype C is present. The readout is the same as it was in the second instance except that the signal for haplotype C is diminished 10 fold in relation to the one for haplotype B. Even at this low relative concentration of haplotype C, it is obvious that it must be present. Error resulting from the 90% detection/chemistry efficiency of haplotype B would not show up in the C channel. Therefore, if it is known that only haplotypes B and C can be present, the detection of any signal in the haplotype C channel means that haplotype C must be present.

Hybridization-Based Approach Using PNAs for SNP-Based Discrimination.

Two-color PNAs can be used for the same type of approach for SNP-based discrimination. PNAs labeled with two different colors are hybridized to either single or double-stranded DNA. The hybridization mixture is washed, cleaned-up, and introduced into the single molecule reader for direct analysis. PNAs generated to hybridize to a specific SNP can be labeled with two different fluorescent moieties. After hybridization, a colored intercalant can be added for a third color, and the labeled DNA can be detected by either being passed by a detector to find the color and position of the different fluorescent labels on the DNA, or simply observed to find the color combination on the labeled DNA.

Primer Extension Using Four-Color Analysis.

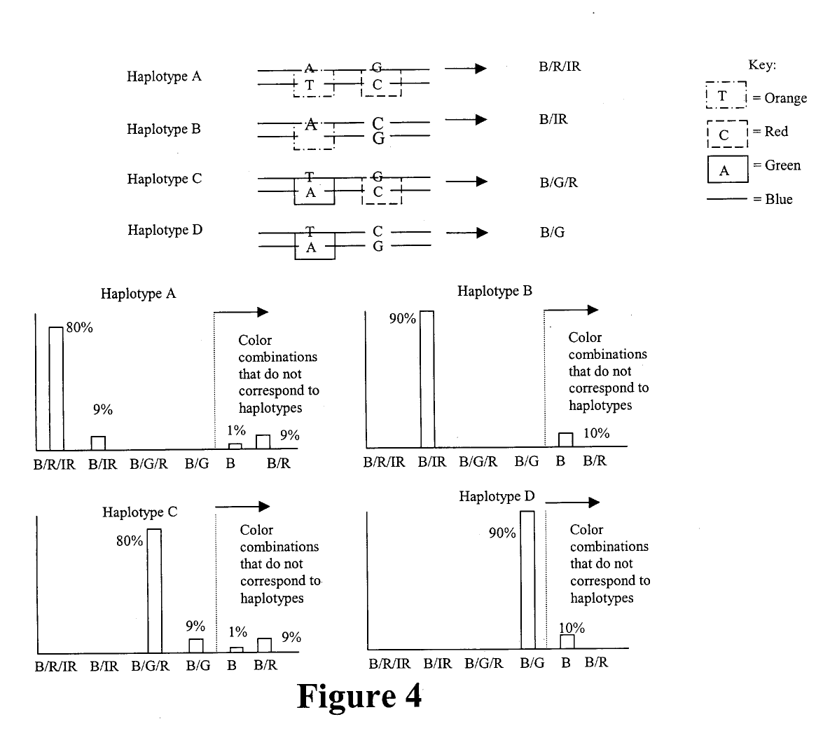

Similar to three color analysis described above, additional information can be obtained by using one additional fluorescent dNTP for SNP haplotype analysis. In addition to fluorescent dUTPs and dCTPs, fluorescent dATPs can be easily obtained. For instance, IR70—dATP (Li-Cor) can be utilized as the fourth color (three colors from the dNTPs and a fourth from the intercalator). In FIG. 4, the different cases of multi-color fluorescent haplotypes can be analyzed. In this schematic T is labeled with a first color, e.g. orange (IR), C is labeled with a second color, e.g. red (R), A is labeled with a third color, e.g. green (G), and the DNA is labeled with an intercalator labeled a fourth color, e.g. blue (B). The assay is set up as the three color analysis presented in FIG. 3. In this way, error from the 90% detection/chemistry efficiency can be differentiated from low level presence of various haplotypes in more situations.

In FIG. 4, the long PCR fragments are hybridized with a SNP-specific primer and extended in the presence of the three dNTPs only. Incorporation of the dNTPs only occurs when there is complementarity between the bases. Assay of the two SNPs allows for four different possible haplotypes, 2 versions. Using three different colored dNTPs for the primer extension and a fourth color for intercalator of the molecules, the four different color combinations allow distinguishing of the different haplotypes.

Haplotype Analysis Using Four-Color Analysis of Four Primer Extended Bases.

The primer extension reaction can also be used with four differently labeled dNTPs. In one example T is color 1 (e.g. orange), C is color 2 (e.g. red), A is color 3 (e.g. blue), and G is color 4 (e.g. green). Haplotype A has T for SNP1 and C for SNP2, haplotype B has A for SNP1 and C for SNP 2, haplotype C has T for SNP1 and G for SNP2, and haplotype D has A for SNP 1 and G for SNP 2. Each dNTP has a different spectrally distinguishable fluorophore.

In this scenario, with a pair of SNPs that allow for four-color spectral discrimination, the use of four color analysis facilitates unambiguous discrimination of the four bases. Haplotype A is blue and orange, haplotype B is blue and red, haplotype C is orange and green, and haplotype D is blue and green. In this example, each of the four haplotypes are determined by a unique color combination.

Limitations of Multi-Color Single Molecule Haplotype Analysis.

There are particular limitations to multi-color analysis of haplotypes using single molecule detection. For instance, the pair of SNPs that are chosen need to be such that the various polymorphisms that are present give rise to four different primer extended products. The methodology would not work in the following scenario. T is labeled with color 1 (e.g. orange) and A is labeled with color 2 (e.g. blue). Haplotype A has T for SNP1 and T for SNP2, haplotype B has T for SNP 1 and A for SNP 2, haplotype C has A for SNP1 and A for SNP2, and haplotype D has A for SNP 1 and T for SNP 2. Therefore, haplotype A is blue, haplotype B is blue and orange, haplotype C is orange, and haplotype D is orange and blue. Haplotypes A and D are indistinguishable by color combination. So, unique color combinations are not generated for the four different haplotypes. In this manner, inefficiencies in single molecule detection would complicate the analysis even further, making haplotype determination impossible for this combination of SNPs. SNPs need to be chosen so that the four different combinations of bases are incorporated upon primer extension of the bases.

Additional Chemistries Used for SNP Assay Detection Using Direct DNA Analysis

One method for SNP detection includes direct hybridization. DNA can be interrogated using different chemistries for multi-color sequence-specific labeling. In the case of oligonucleotides, four different oligonucleotides can be used to hybridize to the sites of interests, assuming that the SNP combination allows for the four oligonucleotides to be assayed to give independent haplotype information.

Competing oligonucleotides labeled with different fluorophores are introduced into the DNA sample and hybridized under stringent conditions that allow competition of the oligonucleotides. The correct match allows the proper haplotype to be determined. This type of analysis can be used with ssDNA and dsDNA with the correct sequence-specific tagging chemistry. The approach can be accomplished with any type of ssDNA or dsDNA sequence-specific tagging chemistry that incorporates a distinguishable label for the detection methodology.

In one example, SNP 1 can be hybridized with an A or a T, and SNP 2 can be hybridized with a C or a G. An oligonucleotide which specifically hybridizes to SNP 1 when it hybridizes to an A is labeled with color 1 (e.g. blue). An oligonucleotide which specifically hybridizes to SNP 1 when it hybridizes to a T is labeled with color 2 (e.g. orange). An oligonucleotide which specifically hybridizes to SNP 2 when it hybridizes to a C is labeled with color 3 (e.g. red). An oligonucleotide which specifically hybridizes to SNP 2 when it hybridizes to a G is labeled with color 4 (e.g. green). Haplotype A has T for SNP1 and C for SNP2, haplotype B has A for SNP1 and C for SNP 2, haplotype C has T for SNP1 and G for SNP2, and haplotype D has A for SNP 1 and G for SNP 2. Haplotype A is blue and orange, haplotype B is blue and red, haplotype C is orange and green, and haplotype D is blue and green. In this example, each of the four haplotypes are determined by a unique color combination.

This method could be used even if the two SNPs hybridized to the same nucleotide. The oligonucleotide is labeled as a unit. Each oligonucleotide will usually only be able to hybridize to one SNP and not the other. Therefore, even if SNP 1 hybridized to A and SNP 2 also hybridized to A, oligo 1, which hybridizes specifically with the “A” SNP 1, would not hybridize with the “A” SNP2 except under the unlikely circumstance that the sequence surrounding the two SNPs was exactly the same.

Confirmation by Assaying the Complementary Strand of DNA.

Confirmation of the SNP haplotypes can be performed through assaying the complementary strand of the DNA. This analysis allows the confirmation of haplotypes using the same mixture of fluorescently labeled dNTPs.

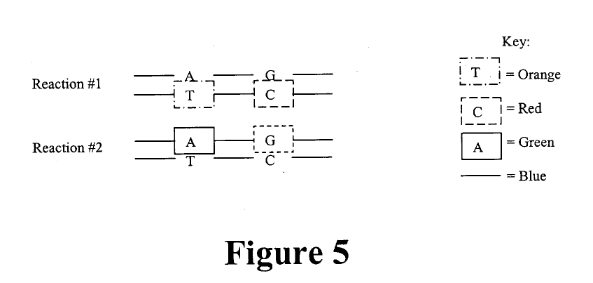

Reaction #1, in FIG. 5, assays the dual color detection of the primer extended products and determines the haplotype to be T and C in a consecutive manner. Reaction #2 is performed in a separate tube and is also assayed using the same fluorescently labeled mixture of dNTPs. A primer extension reaction is performed using the opposite primer extension product with primers that recognize the complementary strand of the region of interest. In this manner, the haplotypes are confirmed using analysis of the opposite strand of DNA. The ability to confirm the haplotypes lowers the rate of false positive and false negative haplotypes.

Confirmation by Rotation of Colors on the dNTPs.

Similar to a confirmation through complementary strand analysis, rotation of the colors of the dNTPs allows for greater confidence in the haplotypes present in the mixture. This method allows for overcoming any errors and differences in incorporations of the four different dNTPs. For instance, a haplotype with A and G in consecutive locations on a strand of DNA may be red and green in one experiment. In a confirmatory reaction, the A and G that are introduced into the reaction mixture may be orange and blue. The presence of red and green in the first experiment and orange and blue in the second experiment indicates a confirmed haplotype in the system.

Assay of a Three SNP Haplotype

A three or four SNP haplotype is more difficult to perform because of the greater number of SNPs that need to be analyzed. In this embodiment, inefficiencies in the chemistry of single molecule analysis require more sophisticated data analysis. With a three SNP haplotype and analysis using three colors, the inefficiencies in single molecule chemistry/detection complicate the analysis.

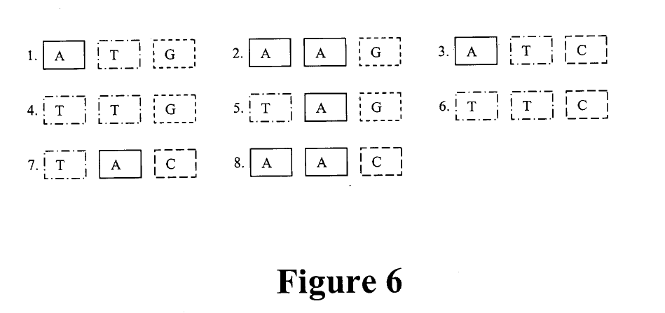

In the case of 100% efficient detection and sequence-specific chemistries, each of the eight possible haplotypes has a unique color signature using four bases that are labeled with different fluorophores. However, since detection chemistries and detection are imperfect due to inefficient chemistries, photobleaching, or inactive fluorophores, these color combinations may be confused, as shown in Table 1, and FIG. 6. In FIG. 6, C is color 1 (represented by a long dashed line), G is color 2 (represented by a short dashed line), T is color 3 (represented by a long dash followed by a short dash), and A is color 4 (represented by a solid line).

Through efficient detection and sequence-specific chemistries, each of the 8 possible haplotypes in FIG. 6 has a unique color signature using four bases that are labeled with different fluorophores. However, since detection chemistries and detection are imperfect due to inefficient chemistries, photobleaching, and inactive fluorophores, these color combinations may be confused. Haplotype 3, through inability to detect the A, would be confused for haplotype 6. Haplotype 3, upon the inability to detect T, would be confused for haplotype 8. Haplotype 1 is redundant with haplotype 5. These would not be able to be distinguished. Other labeling strategies would be necessary to differentiate all of the haplotypes. In a case where haplotype 1, 3, 6, and 8 were in the reaction mixture and the detection/chemistry efficiency was 90%, a snapshot of the haplotyping data is shown in Table 1.

TABLE 1
Relative values of presence of haplotypes in the reaction mixture.

| | Relative number of haplotypes in |
|---|---|
| Haplotype | mixture (arbitrary units) |
| 1 | 72 |
| 2 | (8) |
| 3 | 72 |
| 4 | (8) |
| 5 | 72 |
| | (duplicate of 1) |
| 6 | (8) + 72 |
| 7 | 72 |
| | (duplicate of 3) |
| 8 | (8) + 81 |

In this embodiment of the color scheme, the labeling strategy is ineffective because there is not a unique color scheme to match the haplotypes. The background signals from the different color schemes do not confuse the output signal that much. The background signal representing haplotype 2 is 8, clearly discriminated from the haplotypes that are truly represented in the reaction set. However, if it was not known what haplotypes were present, haplotype 1 would not be able to be distinguished from haplotype 5, and haplotype 3 would not be able to be distinguished from haplotype 7.

This can be overcome in situations where the error associated with the detection and labeling chemistry changed dependent upon the SNP position. The presence of haplotypes 1 and 5 produce error that shows up as being haplotypes 2 and 4. Haplotype 1 shows up as haplotype 2, when it loses label at SNP position 2. Haplotype 5 shows up as haplotype 2 when it loses label at SNP position 1. If this loss of label is different for these positions, the difference in signal showing up as haplotype 2 could be used to differentiate between haplotypes 1 and 5.

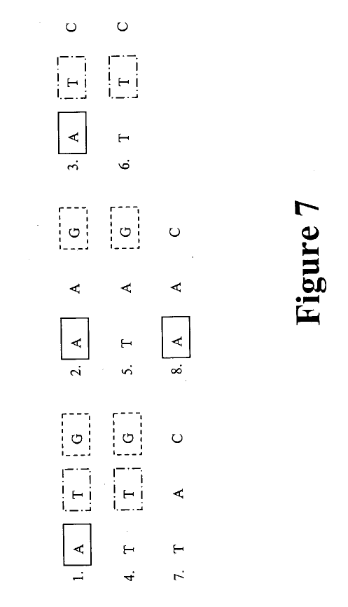

More effective labeling schemes can be used in the assessment of a three SNP haplotype. One possible labeling scheme is the A, T, G are labeled with a different fluorophore and the biallelic nature of SNPs allows the matching of the 8 different possible haplotypes with a unique color scheme. The use of this labeling scheme creates greater “crosstalk” between the different haplotypes as shown in FIG. 7 using the same color scheme as FIG. 6.

Table 2 examines a reaction mixture with haplotypes 1, 3, 6, and 8 in a particular reaction mixture. The detection/chemistry efficiency is at 90%.

TABLE 2
Relative values of presence of haplotypes in the reaction mixture.

| | Relative number of haplotypes in |
|---|---|
| Haplotype | mixture (arbitrary units) |
| 1 | 72 |
| 2 | (8) |
| 3 | 90 + (9) |
| 4 | (8) |
| 5 | (9) |
| 6 | 90 + (9) |
| 7 | (10 + 10 + 1 + 0.1) |
| 8 | 90 |

The background crosstalk is shown in parentheses. Haplotype 7 has the highest background level. Despite this value, the signal-to-noise is still high enough for the haplotypes present in the sample to be determined. Using this tagging scheme the haplotype analysis can be extended to three or four SNPs. The limitation arises from the number of colors that are possible for each of the different SNPs. One distinct color for each SNP is required.

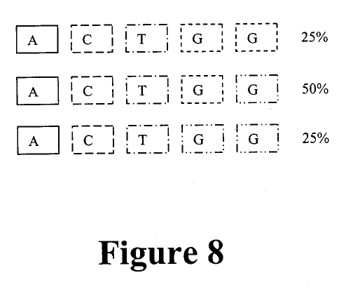

A five SNP haplotype can also be determined through the judicious use of tagging approaches. In the labeling scheme shown in FIG. 8, the first four SNPs, A, C, T, G are labeled with four different colors, color 1 (e.g. blue-represented by a solid line), color 2 (e.g. red—represented by a long dashed line), color 3 (e.g. orange—represented by a long dash followed by a short dash), and color 4 (e.g. green—represented by a short dashed line), respectively. The fifth G is distinguished through the use of mixture tagging approach where the dGTP is represented by a 50/50 population of color 4 (e.g. green) labeled dGTP and color 5 (e.g. purple) labeled dGTP. In this manner, the haplotype is detected through the presence of simultaneous five color detection on one of the fragments of DNA. When two SNPs hybridize specifically to the same nucleotide, a 1:2:1 distribution of both color 1, color 1 and color 2, and both color 2 emerges. Similarly, this approach can be applied to more than five SNPs and its respective haplotype.

Special Considerations for Multi-Color Single Molecule Analysis

For the primer extension example, particularly the four-color assay, the selection of the fluorophores and optical system is particularly important for the successful reading of the single molecule products. The emission spectra of the four fluorophores need to be extremely well separated in order to be able to fully distinguish the individual fluorophores at the single molecule level. The current invention discloses a selection of wavelengths and fluorophores that allow this multi-color analysis to occur.

TABLE 3

| Color | Deoxy nucleotide | Dideoxy nucleotide |
|---|---|---|
| Cascade Blue | dUTP | n/a commercially yet |
| TAMRA | dUTP | all |
| Cy3 | dCTP | n/a commercially yet |
| Cy5 | dCTP | n/a commercially yet |
| IR800 | dATP | all |

Other fluorophores known in the art could also be used.

Special Considerations for Apparatus in Single Molecule Detection Haplotype Analysis

The current invention can be carried out using an apparatus that holds the sample with a slide/coverslip, capillary, or microchip. One embodiment is through the use of a flow-through nanochip such as that described in U.S. Pat. Nos. 6,403,311 and 6,355,420.

PCR is not needed for the analysis if there are enough copies of the genomes to be analyzed in the reaction mixture. The reaction can occur directly on the genomic DNA.

Analysis of more than two populations. The technique of population pooling and subsequent analysis using single molecule genetics can further be applied to correlations of genetics of more than two populations. The methodology of more than two populations allows for complex ethnic analyses where different large founder populations can be compared in mass using single reactions. Different sites along the length of the genomes can be compared using the technique, vastly simplifying the need for different reactions for each individual of the populations.

After pooling the DNA population, in one embodiment of the invention, the DNA is amplified. Insertion/deletion analysis can be performed on the DNA. This is followed by haplotype analysis.

In another embodiment of the invention, the DNA is not amplified after pooling. Direct linear analysis is performed on the DNA and SNP analysis is performed. Then other genetic variations like microsatellites, SNPs, mutations and others can be looked at.

Methods of Analyzing Polymers Using Ordered Label Strategies

The labeled DNA fragments of the invention above can be analyzed using methods disclosed in U.S. Pat. No. 6,403,311. This patent includes details of how to analyze polymers shown below.

A “labeled unit specific marker” as used herein is any unit specific marker in a polymer that identifies a particular unit or units. A labeled unit specific marker includes, for instance, fluorescent markers which are bound to a particular unit or units, proteins, peptides, nucleic acids, polysaccharides, short oligomers, tRNA, etc. that recognize and bind to a particular unit or units and that can be detected by e.g., possessing an intrinsically labeled property or including an extrinsic label or by binding to another detection molecule such as an antibody.

The data obtained from the polymer dependent impulses may be stored in a database, or in a data file, in the memory system of the computer. The data for each polymer may be stored in the memory system so that it is accessible by the processor independently of the data for other polymers, for example by assigning a unique identifier to each polymer.

The information contained in the data and how it is analyzed depends on the number and type of labeled unit specific markers that were caused to interact with the agent to generate signals. For instance if every unit specific marker of a single polymer, each type of unit specific marker (e.g., all the A's of a nucleic acid) having a specific type of label, is labeled then it will be possible to determine from analysis of a single polymer the order of every unit specific marker within the polymer. If, however, only one of the four types of units of a nucleic acid is labeled then more data will be required to determine the complete sequence of the nucleic acid. Additionally, the method of data analysis will vary depending on whether the polymer is single stranded or double stranded or otherwise complexed. Several labeling schemes and methods for analysis using the computer system data produced by those schemes are described in more detail below. The labeling strategies are described with respect to nucleic acids for ease of discussion. Each of these strategies, however, is useful for labeling all polymers.

Several different strategies of labeling are possible, involving permutations of different types of units labeled, different percentage of units labeled, and single-stranded or double-stranded labeling. Set forth below are examples of labeling strategies useful according to the invention. The invention is, however, not limited to the exemplary details provided below. The labeling methods described herein and data obtained from such methods are described with reference to DNA to simplify the discussion. The invention, however, is not limited to methods of analyzing DNA, but rather may be utilized with any type of polymer which is composed of individual monomeric units. It will be clear to those of ordinary skill in the art that when the description below refers to DNA or nucleic acids, any polymer many be substituted, and when the description refers to a nucleotide, a base or specifically A, C, T, or G, these terms may be substituted with the particular monomeric units of the desired polymer. For instance, the polymer may be a peptide, and in that case the monomeric units is an amino acid. The simplest labeling scheme involves the labeling of all four nucleotides with different labels. Labeling schemes in which three, two, or even one unit are labeled, or wherein various combinations of units are labeled using unit specific markers which span multiple nucleotides also possible.

The distance between nucleotides can be determined in several ways. Firstly, the polymer and the station may be moved relative to one another in a linear manner and at a constant rate of speed such that a single unit specific marker of the nucleic acid molecule will pass the station at a single time interval. If two time intervals elapse between detectable signals then the unlabeled nucleotide which is not capable of producing a detectable signal is present within that position. This method of determining the distance between unit specific markers is discussed in more detail below in reference to random one base labeling. Alternatively the polymer and the station may be caused to interact with one another such that each unit specific marker interacts simultaneously with a station to produce simultaneous detectable signals. Each detectable signal generated occurs at the point along the polymer where the unit specific marker is positioned. The distance between the detectable signals can be calculated directly to determine whether an unlabeled unit specific marker is positioned anywhere along the nucleic acid molecule.

In another labeling scheme, the random one nucleotide labeling scheme also may be used. In this method, distance information which is obtained by either population analysis and/or instantaneous rate of DNA movement is used to determine the number of nucleotides separating two labeled nucleotides. Analysis of four differently labeled target molecules yields the complete sequence.

There are two methods of determining the distance between bases. One requires determining the instantaneous rate of DNA movement, which is readily calculated from the duration of energy transfer or quenching for a particular label. Another involves analyzing a population of target DNA molecules and its corresponding Gaussian distance distributions.

The instantaneous rate method, involves a determination of distance separation based on the known instantaneous rate of DNA movement (v) multiplied by the time of separation between signals (t). Instantaneous rate is found by measuring the time that it takes for a labeled nucleotide to pass by the interaction station. Since the length of the concentrated area of agent (d) is known (through calibration and physical measurement of the localized region of the agent, e.g., the thickness of a concentrated donor fluorophore area), the rate is simply v=d/t. Analysis of raw data demonstrating changes in energy emission patterns resulting from sequential detectable signals when plotted produces a curve which from left to right shows two energy intensity decreases, followed by two energy intensity increases. The plateau from the first energy intensity decrease (denoted t.₁) is double that of the second plateau (t.₂). The length of the interaction station is given as 51 Å. From this given information, the number of labeled nucleotides is known. Furthermore, the distance of separation of the two is determined by relating the rate of DNA movement to the time of the donor intensity plateaus.

The number of labeled nucleotides is simply denoted by the number of intensity decreases. If there are two intensity decreases, there must be two detectable labels on the DNA. To determine the distance of base separation, it is necessary to know the instantaneous rate of DNA movement, which is found by knowing the time for one labeled nucleotide to cross the localized region of the agent and the length of the localized region of the agent. The length of the localized region of the agent is given as 51 Å. The time for one labeled nucleotides crossing the localized region of the agent is bounded by the first intensity decrease and the first intensity increase (denoted as the gray shaded region, 7.5 s). The rate of DNA movement is 6.8 Å/s. The base separation is derived from the time separating the labeled nucleotides (t.sub.1=5 s) multiplied by the rate (6.8 Å), which is equal to 10 base pairs. As a means of cross-verification, 51 Å-t₂ v also yields the base separation.

In the population method the entire population of labeled nucleotide is considered. Knowledge of the length of the localized region of the agent and instantaneous rate, as required for the rate method, is not necessary. Use of population analyses statistically eliminates the need for precision measurements on individual nucleic acid molecules.

An example of population analyses using five nucleic acid molecules each traversing a nanochannel is described below. Five molecules representing a population of identical DNA fragments are prepared. In a constant electric field, the time of detection between the first and second labeled nucleotide should be identical for all the DNA molecules. Under experimental conditions, these times differ slightly, leading to a Gaussian distribution of times. The peak of the Gaussian distribution is characteristic of the distance of separation (d) between two labeled nucleotides.

An additional example utilizing a population of one nucleotide randomly labeled nucleic acid molecule (six molecules represent the population) further illustrates the concept of population analysis and the determination of distance information. The nucleic acid is end-labeled to provide a reference point. With enough nucleic acid molecules, the distance between any two A's can be determined. Two molecules, when considered as a sub-population, convey the base separation molecules, distributions of 4 and 6 base separations are created. Extending the same logic to rest of the population, the positions of all the A's on the DNA can be determined. The entire sequence is generated by repeating the process for the other three bases (C, G, and T).

In addition to labeling all of one type of unit specific marker in the above-described examples, it is possible to use various labeling schemes where not every nucleotide of the nucleotides or markers to be labeled is labeled. With a large population of randomly labeled fragments, the distance between every successive A on the target DNA can be found. The end labels serve to identify the distance between the ends of the DNA and the first A. Repeating the same analysis for the other nucleotides generates the sequence of the 16-mer by compiling the data to identify the position of all of the As within that population of nucleic acid molecules. These steps can then be repeated using unit specific markers for the other nucleotides in the population of nucleic acids. The advantages of using such a method includes lack of steric effects and ease of labeling. This type of labeling is referred to as random labeling. A polymer which is “randomly labeled” is one in which fewer than all of a particular type of unit specific marker are labeled. It is unknown which unit specific markers of a particular type of a randomly labeled polymer are labeled.

A similar type of analysis may be performed by labeling each of the four nucleotides incompletely but simultaneously within a population. For instance, each of the four nucleotides may be partially labeled with its own unit specific marker which gives rise to a different physical characteristic, such as color, size, etc. This can be accomplished to generate a data set containing information about all of the nucleotides from a single population analysis. For instance the method may be accomplished by partially labeling two nucleotide pairs at one time. Two nucleotide labeling is possible through the lowering of steric hindrance effects by using unit specific markers which recognize the two nucleotides of a nucleic acid strand and which contain a label such as a single fluorescent molecule. Goodwin et al., Nucleic Acids Research, 21(4):803-806, 1993 and Harding and Keller, Trends Biotechnol, 10(1-2):55-57, 1992, have demonstrated that large fluorescent nucleic acid molecules with two of the nucleotides completely labeled are possible to achieve. The average size of the molecules studied were 7 KB. Partial labeling of three nucleotides is also possible. For instance, each of three nucleotides is partially labeled with a different unit specific marker. In this case, a population of single stranded nucleic acid molecules which are partially labeled with three specific nucleotide pair combinations is generated and can be analyzed.

The methods of the invention can also be achieved using a double stranded nucleic acid. In a double stranded nucleic acid, when a single nucleotide on two of the strands is labeled, information about two nucleotides becomes available for each of the strands. For instance, in the random and partial labeling of A's, knowledge about the A's and T's becomes available. A labeling strategy in which two differently labeled nucleic acid samples are prepared can be used. The first sample can have two non-complimentary nucleotides randomly labeled with the same fluorophore. Non-complimentary pairs of nucleotides are AC, AG, TC, and TG. The second sample can have one of its nucleotides randomly labeled. The nucleotide chosen for the second sample may be any one of the four nucleotides. In the example provided, the two non-complimentary nucleotides are chosen to be A and C, and the single nucleotide is chosen to be A. Two samples are prepared, one with labeled A's and C's and another with labeled A's. The nucleic acid is genomically digested, end labeled, purified, and analyzed. Such procedures are well-known to those of ordinary skill in the art. The information from each fragment is sorted into one of two complimentary strand groups. Sorting the information allows the population analysis to determine the positions of all the desired nucleotides. The first group of data provides known positions of all the A's and C's on one strand. The second group of data provides known positions of all of the A's. The combination of these two data sets reveals the position of all of the A's and C's on one strand. The same procedure may be applied to the complimentary strand to determine the positions of the A's and C's on that strand. The resultant data reveals the entire sequence for both strands of the nucleic acid, based on the assumption that the strand includes the complimentary nucleotide pairs of A and C (A:T and C:G). To cross-verify the sequence, the process can be repeated for the other pairs of non-complimentary nucleotides such as TG, TC and AG.

A single-stranded two-nucleotide labeling scheme also can be performed on double stranded DNA when two of the nucleotides on one strand of DNA are fully replaced by labeled nucleotides. To reduce the steric constraints imposed by two extrinsically labeled nucleotides while preserving the theory behind two-nucleotide labeling, it is possible to label one nucleotide fully on each of the complementary strands to achieve the same end. This method involves using double-stranded DNA in which each strand is labeled with a different label. Six differently labeled duplex DNA sets will produce a data set which is adequate to provide sequence information. Each complementary strand of DNA should have one of the nucleotides labeled. In each of the duplex DNA sets, the equivalent of two different nucleotides (possible combinations are AC, AG, AT, CG, CT, GT) are labeled. When both complementary strands have the adenines labeled, this is equivalent to the combination AT. In duplex two-nucleotide labeling, the advantage is that only one nucleotide on each strand is labeled, allowing longer labeled strands to be synthesized as compared to two-nucleotide labeling on single-stranded DNA. In practice, it has been shown that synthesis of DNA fragments with one nucleotide completely labeled can be achieved with lengths much greater than 10 kb (Goodwin et al., Nucleic Acids Research, 21(4):803-806, 1993 and Harding and Keller, Trends Biotechnol, 10(l-2):55-57, 1992).

By including more than one physical characteristic into the label, the simultaneous and overlapping reading of the nucleic acid within the same temporal frame may provide more accurate and rapid information about the positions of the labeled nucleotides than when only a single physical characteristic is included. For instance, each of the nucleotides can include a double or triple Each of the fluorophores can be detected separately to provide distinct readings form the same sample.

In addition to the various combinations of single nucleotide labeling methods, two or more adjacent nucleotides may be specifically labeled. As described above a unit specific marker includes markers which are specific for individual nucleotides as well as markers which are specific for multiple nucleotides. Multiple nucleotides include two or more nucleotides which may or may not be adjacent. For instance, if a unit specific marker is a complex of protein, the complex of proteins may interact with specific nucleotides that are adjacent to one another or which are separated by random nucleotides. This type of analysis is particularly useful because detection of the signal requires less resolution than with single nucleotide analysis. The more complex the analysis, the greater resolution of the system. Resolution as used herein refers to the number of nucleotides which can be resolved by the appropriate signal detection method used.

Preferably the signal detection method includes methods such as nanochannel analysis, near-field scanning microscopy, atomic force microscopy, scanning electron microscopy, waveguide structures, etc.

The greater the number of nucleotides a unit specific marker spans and recognizes, the more amenable that unit specific marker is to low resolution means of detection. For any given number of nucleotide-spanning markers, the number of different unit specific markers which can be used is defined by the formula 4″, where n is the number of nucleotides detected by the unit specific marker. A unit specific marker which spans two nucleotides would be specific for one of 16 combinations of nucleotide pairs. These include, AC, AG, AT, AA, CC, CA, CG, CT, GA, GG, GC, GT, TA, TC, TG, and TT. A unit specific marker which spans three nucleotides would be specific for one of a combination of 64 three nucleotide pairs combinations. More than three nucleotide pairs combinations may also be used, and the number would increase according to the above formula. Using these types of unit specific markers, nucleotide sequence information can be reconstructed through a number of different means. The information generated from the reconstruction of the unit specific markers is not limited to the generation of sequence information, but additionally can be used to unambiguously identify fragments, provide the specific number of that combination of nucleotides found within the sequence, etc.

Various combinations of triplet unit specific markers bound to a nucleic acid molecule can be deciphered and analyzed using these methods. Without knowing the precise location of the triplet unit specific markers on the nucleic acid, the specificity given to a bound nucleic acid fragment is given as N/4ⁿ where N is the number of nucleotides in the fragment of target nucleic acid and n is the number of bound sites on the nucleic acid. The longer the strand of nucleic acid, the lower the specificity of the particular system. The specificity of the bound unit specific markers can be increased by determining the precise location of the triplet unit specific markers. In this case, the specificity is increased to ¼ⁿ which is the same as if an N-mer were bound to the target strand of nucleic acid.

The simplest method to determine the sequence of the nucleic acid molecule from the set of triplet unit specific markers is to examine two triplet 1 unit specific markers one time until all 64 unit specific markers are examined. If one of the triplet unit specific markers is kept constant during the analysis, the analysis is simplified. In one example, a short stretch of nucleic acid is analyzed using two triplet unit specific markers. The triplet unit specific markers are CGX and GXX. Using these markers, the two based positions after the first ACG triplet can be determined. Using the 63 different triplets together with the initial fragment ACG, information about flanking nucleotides and the contiguous sequence of the intervening nucleotides between the ACGs can be determined.

Using these methods of sequence analysis, problems which occur in other types of hybridization, etc. analysis are avoided. For instance, repeated sequences such as the Alu repeats in the human genome create analysis problems using hybridization sequencing methods. Such problems are avoided using the methods described herein. Using the methods described herein the number of repeats can be simply counted by the different triplets bound in each of the states. Hybridization sequencing analysis does not allow the determination of linear order or number of probes found between two probe sequences. The linear order and the precise quantitation of the number of probes bound allows an additional order of information which bypasses the difficulties faced in sequencing by hybridization. The methods of the invention are thus rapid and straightforward.

The method using triplet, etc. unit specific markers does not need to be performed sequentially. For instance, several triplets may be assayed simultaneously to provide an even more rapid method of analysis. The only limitation in simultaneous analysis is that none of the triplet unit specific markers used simultaneously should overlap one another. Therefore, the choice of one particular triplet sequence precludes the simultaneous use of triplet sequences which would overlap with that sequence. For example if the triplet sequence ACG is selected for analysis, 4 of the 64 sets of triplets may not be used during simultaneous analysis with this triplet. These include XXA, XAC, GXX, and CGX. Mathematically, the maximum number of fragments which a triplet label can preclude simultaneous probing with is determined by the following equation:

2[σ4²+4¹] or generally 2[σ4ⁿ⁻¹+4ⁿ⁻²4¹]

where n is the number of nucleotides spanned by the labels. The sum is that a maximum of 40 fragments are precluded from simultaneous assay with the originally selected ACG triplet. Therefore, a total of 24 different fragments may be assayed at one time.

Double stranded nucleic acid analysis also may be accomplished using direction specific labels. Direction specific labels allow for discrimination between a combination of nucleotides such as ACG triplet on either strand. In the case of direction specific labels, the reversal of the center bound label shows that it is a label bound on the opposite strand. The labels have 5′ to 3′ or 3′ to 5′ directionality.

One use for the methods of the invention is to determine the sequence of units within a polymer. Identifying the sequence of units of a polymer, such as a nucleic acid, is an important step in understanding the function of the polymer and determining the role of the polymer in a physiological environment such as a cell or tissue. The sequencing methods currently in use are slow and cumbersome. The methods of the invention are much quicker and generate significantly more sequence data in a very short period of time.

The analysis methods described herein may be linear or non linear. The methods for generating sequence information based on data obtained from partially labeled polymers can be applied to data obtained by any method that produces polymer dependent impulses. The reconstruction of the sequence of the polymer from this type of data is an integral aspect of the invention. As long as the data is obtained by a method for detecting the polymer dependent impulses, whether it is obtained in a linear manner or not, the data may be analyzed according to the methods of the invention.

The signals may be detected sequentially or simultaneously. As used herein signals are detected “sequentially” when signals from different unit specific markers of a single polymer are detected spaced apart in time. Not all unit specific markers need to be detected or need to generate a signal to detect signals “sequentially.” When the unit specific markers are sequentially exposed to the station the unit specific marker and the station move relative to one another. As used herein the phrase “the unit specific marker and the station move relative to one another” means that either the unit specific marker and the station are both moving or only one of the two is moving and the other remains stationary at least during the period of time of the interaction between the unit specific marker and the station. The unit specific marker and the station may be moved relative to one another by any mechanism. For instance the station may remain stationary and the polymer may be drawn past the station by an electric current. Other methods for moving the polymer include but are not limited to movement resulting from a magnetic field, a mechanical force, a flowing liquid medium, a pressure system, a gravitational force, and a molecular motor such as e.g., a DNA polymerase or a helicase when the polymer is DNA or e.g., myosin when the polymer is a peptide such as actin. In one example, the polymer is moved hydrodynamically, e.g., the sample is present in a solution which flows past the detector by being entrained in the fluid flow stream. The fluid is driven through using either pressure or a vacuum.

The movement of the polymer may be assisted by the use of a channel, groove or ring to guide the polymer. Alternatively the station may be moved and the polymer may remain stationary. For instance the station may be held within a scanning tip that is guided along the length of the polymer.

In another embodiment signals are detected simultaneously. As used herein signals are “detected simultaneously” by causing a plurality of the labeled unit specific markers of a polymer to be exposed to a station at once. The plurality of the unit specific markers can be exposed to a station at one time by using multiple interaction sites. Signals can be detected at each of these sites simultaneously. For instance multiple stations may be localized at specific locations in space which correspond to the unit specific markers of the polymer. When the polymer is brought within interactive proximity of the multiple stations signals will be generated simultaneously. This may be embodied, for example, in a linear array of stations positioned at substantially equivalent distances which are equal to the distance between the unit specific markers. The polymer may be positioned with respect to the station such that each unit specific marker is in interactive proximity to a station to produce simultaneous signals.

Multiple polymers can be analyzed simultaneously by causing more than one polymer to move relative to respective stations at one time. The polymers may be similar or distinct. If the polymers are similar, the same or different unit specific markers may be detected simultaneously.

A preferred method for moving a polymer past a station according to the invention utilizes an electric field. An electric field can be used to pull a polymer through a channel because the polymer becomes stretched and aligned in the direction of the applied field as has previously been demonstrated in several studies (Bustamante, Annu. Rev. Biophys. Chem., 20:415-46, 1991; Gurrieri et al., Biochemistry, 29(13):3396-3401, 1990; and Matsumoto et al., J. Mol Biol., 152:501-516, 1981).

Another method for moving a polymer past a station involves the use of a molecular motor. A molecular motor is a device which physically interacts with the polymer and pulls the polymer past the station. Molecular motors include but are not limited to DNA and RNA polymerases and helicases. DNA polymerases have been demonstrated to function as efficient molecular motors. Preferably the internal diameters of the regions of the polymerase which clamp onto the DNA is similar to that of double stranded DNA. Furthermore, large amounts of DNA can be able to be threaded through the clamp in a linear fashion. Molecular motors are described in more detail in U.S. Pat. No. 6,210,896, the entire contents of which is hereby incorporated by reference.

The overall structure of the .beta.-subunit of DNA polymerase III holoenzyme is 80 A in diameter with an internal diameter of .about.35 Å. In comparison, a full turn of duplex B-form DNA is .about.34 Å. The beta subunit fits around the DNA, in a mechanism referred to as a sliding clamp mechanism, to mediate the processive motion of the holoenzyme during DNA replication. It is well understood that the .beta.-subunit encircles DNA during replication to confer processivity to the holoenzyme (Bloom et al., J. Biol. Chem., 271:30699-708, 1996; Fu et al., EMBO J., 15:4414-22, 1996; Griep, Anal. Biochem., 232:180-9, 1995; Herendeen and Kelly, Cell, 84:5-8, 1996; Naktinis et al., Cell, 84(1):137-145, 1996; Paz-Elizur et al., J. Biol. Chem., 271:2482-90, 1996 and Skaliter et al., J. Biol. Chem., 271:2491-6, 1996). Because the sliding clamp is the mechanism of processivity for a polymerase, it necessarily means that large amounts of DNA are threaded through the clamp in a linear fashion. Several kilobases are threaded through the clamp at one time (Kornberg and Baker, DNA Replication, W. H. Freeman, New York, 1991).

The detectable signal (polymer dependent impulse) is produced at a detection station, where a portion of the polymer to be detected (e.g. the unit specific marker) is exposed, in order to produce a signal or polymer-dependent impulse. When the interaction between the unit specific marker and the station produces a polymer-dependent impulse the station is a “signal generation station”. One type of signal generation station is an interaction station. As used herein an “interaction station or site” is a region where a unit specific marker of the polymer interacts with an agent and is positioned with respect to the agent in close enough proximity whereby they can interact. The interaction station for fluorophores, for example, is that region where they are close enough so that they energetically interact to produce a signal.

The interaction station in one embodiment is a region of a nanochannel where a localized agent, such as an acceptor fluorophore, attached to the wall forming the channel, can interact with a polymer passing through the channel. The point where the polymer passes the localized region of agent is the interaction station. As each labeled unit specific marker of the polymer passes by the agent a detectable signal is generated. The agent may be localized within the region of the channel in a variety of ways. For instance the agent may be embedded in the material that forms the wall of the channel or the agent may be attached to the surface of the wall material. Alternatively the agent may be a light source which is positioned a distance from the channel but which is capable of transporting light directly to a region of the channel through a waveguide. An apparatus may also be used in which multiple polymers are transported through multiple channels. These and other related embodiments of the invention are discussed in more detail below. The movement of the polymer may be assisted by the use of a groove or ring to guide the polymer.

Other arrangements for creating interaction stations are embraced by the invention. For example, a polymer can be passed through a molecular motor tethered to the surface of a wall or embedded in a wall, thereby bringing unit specific markers of the polymer sequentially to a specific location, preferably in interactive proximity to a proximate agent, thereby defining an interaction station. A molecular motor is a biological compound such as polymerase, helicase, or actin which interacts with the polymer and is transported along the length of the polymer past each unit specific marker. Likewise, the polymer can be held from movement and a reader can be moved along the polymer, the reader having attached to it the agent. For instance the agent may be held within a scanning tip that is guided along the length of the polymer. Interaction stations then are created as the agent is moved into interactive proximity to each unit specific marker of the polymer.

The agent that interacts with the unit specific marker of the polymer at the interaction station is selected from the group consisting of electromagnetic radiation, a quenching source, and at fluorescence excitation source. “Electromagnetic radiation” as used herein is energy produced by electromagnetic waves. Electromagnetic radiation may be in the form of a direct light source or it may be emitted by a light emissive compound such as a donor fluorophore. “Light” as used herein includes electromagnetic energy of any wavelength including visible, infrared and ultraviolet.

As used herein, a quenching source is any entity which alters or is capable of altering a property of a light emitting source. The property which is altered can include intensity fluorescence lifetime, spectra, fluorescence, or phosphorescence.

A fluorescence excitation source as used herein is any entity capable of fluorescing or giving rise to photonic emissions (i.e. electromagnetic radiation, directed electric field, temperature, fluorescence, radiation, scintillation, physical contact, or mechanical disruption.) For instance, when the unit specific marker is labeled with a radioactive compound the radioactive emission causes molecular excitation of an agent that is a scintillation layer which results in fluorescence.

When a unit specific marker of the polymer is exposed to the agent the interaction between the two produces a signal. The signal provides information about the polymer. For instance, if all unit specific markers of a particular type, e.g., all of the alanines, of a protein polymer are labeled (intrinsic or extrinsic) with a particular light emissive compound then when a signal characteristic of that light emissive compound is detected upon interaction with the agent the signal signifies that an alanine residue is present at that particular location on the polymer. If each type of unit specific marker e.g., each type of amino acid is labeled with a different light emissive compound having a distinct light emissive pattern then each amino acid will interact with the agent to produce a distinct signal. By determining what each signal for each unit specific marker of the polymer is, the sequence of units can be determined.

The interaction between the unit specific marker and the agent can take a variety of forms, but does not require that the unit specific marker and the agent physically contact one another. Examples of interactions are as follows. A first type of interaction involves the agent being electromagnetic radiation and the unit specific marker of the polymer being a light emissive compound (either intrinsically or extrinsically labeled with a light emissive compound). When the light emissive unit specific marker is contacted with electromagnetic radiation (such as by a laser beam of a suitable wavelength or electromagnetic radiation emitted from a donor fluorophore), the electromagnetic radiation causes the light emissive compound to emit electromagnetic radiation of a specific wavelength. The signal is then measured. The signal exhibits a characteristic pattern of light emission and thus indicates that a particular labeled unit specific marker of the polymer is present. In this case the unit specific marker of the polymer is said to “detectably affect the emission of the electromagnetic radiation from the light emissive compound.”

A second type of interaction involves the agent being a fluorescence excitation source and the unit specific marker of the polymer being a light emissive or a radioactive compound. When the light emissive unit specific marker is contacted with the fluorescence excitation source, the fluorescence excitation source causes the light emissive compound to emit electromagnetic radiation of a specific wavelength. When the radioactive unit specific marker is contacted with the fluorescence excitation source, the nuclear radiation emitted from the unit specific marker causes the fluorescence excitation source to emit electromagnetic radiation of a specific wavelength. The signal then is measured.

A variation of these types of interaction involves the presence of a third element of the interaction, a proximate compound which is involved in generating the signal. For example, a unit specific marker may be labeled with a light emissive compound which is a donor fluorophore and a proximate compound can be an acceptor fluorophore. If the light emissive compound is placed in an excited state and brought proximate to the acceptor fluorophore, then energy transfer will occur between the donor and acceptor, generating a signal which can be detected as a measure of the presence of the unit specific marker which is light emissive. The light emissive compound can be placed in the “excited” state by exposing it to light (such as a laser beam) or by exposing it to a fluorescence excitation source.

Another interaction involves a proximate compound which is a quenching source. In this instance, the light emissive unit specific marker is caused to emit electromagnetic radiation by exposing it to light. If the light emissive compound is placed in proximity to a quenching source, then the signal from the light emissive unit specific marker will be altered.

A set of interactions parallel to those described above can be created wherein, however, the light emissive compound is the proximate compound and the unit specific marker is either a quenching source or an acceptor source. In these instances the agent is electromagnetic radiation emitted by the proximate compound, and the signal is generated, characteristic of the interaction between the unit specific marker and such radiation, by bringing the unit specific marker in interactive proximity with the proximate compound.

The mechanisms by which each of these interactions produces a detectable signal is known in the art. For exemplary purposes the mechanism by which a donor and acceptor fluorophore interact according to the invention to produce a detectable signal including practical limitations which arc known to result from this type of interaction and methods of reducing or eliminating such limitations is set forth below.

Another preferred method of analysis of the invention involves the use of radioactively labeled polymers. The type of radioactive emission influences the type of detection device used. In general, there are three different types of nuclear emission including alpha, beta, and gamma radiation. Alpha emission cause extensive ionization in matter and permit individual counting by ionization chambers and proportional counters, but more interestingly, alpha emission interacting with matter may also cause molecular excitation, which can result in fluorescence. The fluorescence is referred to as scintillation. Beta decay which is weaker than alpha decay can be amplified to generate an adequate signal. Gamma radiation arises from internal conversion of excitation energy. Scintillation counting of gamma rays is efficient and produces a strong signal. Sodium iodide crystals fluoresce with incident gamma radiation.

A “scintillation” layer or material as used herein is any type of material which fluoresces or emits light in response to excitation by nuclear radiation. Scintillation materials are well known in the art. Aromatic hydrocarbons which have resonance structures are excellent scintillator. Anthracene and stilbene fall into the category of such compounds. Inorganic crystals are also known to fluoresce. In order for these compounds to luminesce, the inorganic crystals must have small amounts of impurities, which create energy levels between valence and conduction bands. Excitation and de-excitation can therefore occur. In many cases, the de-excitation can occur through phosphorescent photon emission, leading to a long lifetime of detection. Some common scintillator include NaI (T1), ZnS (Ag), anthracene, stilbene, and plastic phosphors.

Many methods of measuring nuclear radiation are known in the art and include devices such as cloud and bubble chamber devices, constant current ion chambers, pulse counters, gas counters (i.e., Geiger-Muller counters), solid state detectors (surface barrier detectors, lithium-drifted detectors, intrinsic germanium detectors), scintillation counters, Cerenkov detectors, etc.

Analysis of the radiolabeled polymers is identical to other means of generating polymer dependent impulses. For example, a sample with radiolabeled A's can be analyzed by the system to determine relative spacing of A's on a sample DNA. The time between detection of radiation signals is characteristic of the polymer analyzed. Analysis of four populations of labeled DNA (A's, C's, G's, T's) can yield the sequence of the polymer analyzed. The sequence of DNA can also be analyzed with a more complex scheme including analysis of a combination of dual labeled DNA and singly labeled DNA. Analysis of a and C labeled fragment followed by analysis of an A labeled version of the same fragment yields knowledge of the positions of the A's and C's. The sequence is known if the procedure is repeated for the complementary strand. The system can further be used for analysis of polymer (polypeptide, RNA, carbohydrates, etc.), size, concentration, type, identity, presence, sequence and number.

The methods described above can be performed on a single polymer or on more than one polymer in order to determine structural information about the polymer.

A “detectable signal” as used herein is any type of signal or polymer dependent impulse which can be sensed by conventional technology. The signal produced depends on the type of station as well as the unit specific marker and the proximate compound if present. In one embodiment the signal is electromagnetic radiation resulting from light emission by a labeled (intrinsic or extrinsic) unit specific marker of the polymer or by the proximate compound. In another embodiment the signal is fluorescence resulting from an interaction of a radioactive emission with a scintillation layer. The detected signals may be stored in a database for analysis. One method for analyzing the stored signals is by comparing the stored signals to a pattern of signals from another polymer to determine the relatedness of the two polymers. Another method for analysis of the detected signals is by comparing the detected signals to a known pattern of signals characteristic of a known polymer to determine the relatedness of the polymer being analyzed to the known polymer. Comparison of signals is discussed in more detail below.

More than one detectable signal may be detected. For instance a first individual unit specific marker may interact with the agent or station to produce a first detectable signal and a second individual unit specific marker may interact with the agent or station to produce a second detectable signal different from the first detectable signal. This enables more than one type of unit specific marker to be detected on a single polymer.

Once the signal is generated it can then be detected. The particular type of detection means will depend on the type of signal generated which will depend on the type of interaction which occurs between the unit specific marker and the agent. Many interactions involved in the method of the invention will produce an electromagnetic radiation signal. Many methods are known in the art for detecting electromagnetic radiation signals, including two- and three-dimensional imaging systems. These and other systems are described in more detail in PCT Patent Application WO 98/35012 and U.S. Pat. No. 6,355,420.

Other interactions involved in the method will produce a nuclear radiation signal. As a radiolabel on a polymer passes through the defined region of detection, such as the station, nuclear radiation is emitted, some of which will pass through the defined region of radiation detection. A detector of nuclear radiation is placed in proximity of the defined region of radiation detection to capture emitted radiation signals. Many methods of measuring nuclear radiation are known in the art including cloud and bubble chamber devices, constant current ion chambers, pulse counters, gas counters (i.e., Geiger-Muller counters), solid state detectors (surface barrier detectors, lithium-drifted detectors, intrinsic germanium detectors), scintillation counters, Cerenkov detectors, etc.

Other types of signals generated are well known in the art and have many detections means which are known to those of skill in the art. Among these include opposing electrodes, magnetic resonance, and piezoelectric scanning tips. Opposing nanoelectrodes can function by measurement of capacitance changes. Two opposing electrodes create all area of energy storage, which is effectively between the two electrodes. It is known that the capacitance of two opposing electrodes change when different materials are placed between the electrodes. This value is known as a dielectric constant. Changes in the dielectric constant can be measured as a change in the voltage across the two electrodes. In the present example, different nucleotide bases or unit specific markers of a polymer may give rise to different dielectric constants. The capacitance changes as the dielectric constant of the unit specific marker of the polymer per the equation: C═KCₒ, where K is the dielectric constant and CO is the capacitance in the absence of any bases. The voltage deflection of the nanoelectrodes is then outputted to a measuring device, recording changes in the signal with time.

A nanosized NMR detection device can be constructed to detect the passage of specific spin-labeled polymer unit specific markers. The nanosized NMR detection device consists of magnets which can be swept and a means of irradiating the polymer with electromagnetic energy of a constant frequency (this is identical to holding the magnetic field constant while the electromagnetic frequency is swept). When the magnetic field reaches the correct strength, the nuclei absorb energy and resonance occurs. This absorption causes a tiny electric current to flow in an antenna coil surrounding the sample. The signal is amplified and output to a recording device. For known labeled compounds, the time of detection is much faster than current means of NMR detection where a full spectra of the compound in question is required. Known labeled unit specific markers of polymers have known chemical shifts in particular regions, thereby eliminating the need to perform full spectral sweeps, lowering the time of detection per base to micro or milliseconds.

A nanoscale piezoelectric scanning tip can be used to read the different unit specific markers of the polymer based on physical contact of the different polymer unit specific markers with the tip. Depending on the size and shape of the polymer unit specific marker, different piezoelectric signals are generated, creating a series of unit specific marker dependent changes. Labels on unit specific markers are physically different than native units and can create a ready means for detection via a piezoelectric scanning tip. Upon contact of a polymer unit specific marker with the tip, the piezoelectric crystals change and give rise to a current which is outputted to a detection device. The amplitude and duration of the current created by the interaction of the polymer unit specific marker and the tip is characteristic of the polymer unit specific marker.

In one preferred type of linear analysis, the labeled polymer is fixed in a relative position to a station by a nanochannel, such that as the labeled polymer passes the station signals arising from the interaction between the station and the labeled polymer are spatially confined. The channels preferably correspond to the diameter of the labeled polymer and fix the DNA relative to an imaging system which is able to capture many emissions from the labeled polymer over an integrated period of time. The method is specific for the analysis of intensities of individual molecules. The nanochannel system is provided as an example and is discussed in more detail below. Any means can be used to fix the labeled polymers in a dimension for analysis by an optical method capable of analyzing the signals over time. Examples of devices which are capable of positioning labeled polymers for analysis include nanochannel arrays, integrated nanofabricated waveguides, and various lattices.

Molecular Motors

The analysis described above can be performed through the use of molecular motors as described in U.S. Pat. No. 6,210,896, described briefly below.

The methods and products of the invention are useful for determining structural information about a polymer in a similar manner to the linear analysis methods described in WO 98/35012 and U.S. Pat. No. 6,355,420. Thus in one aspect, the methods of the invention can be used to identify one, some, or all of the units of the polymer. This is achieved by identifying the type of individual unit and its position on the backbone of the polymer by determining whether a signal detected at that particular position on the backbone is characteristic of the presence of a particular labeled unit.

In one aspect the invention is a method for analyzing a polymer. The method includes the steps of exposing a plurality of individual units of a polymer to an agent selected from the group consisting of an electromagnetic radiation source, a quenching source, and a fluorescence excitation source by causing a molecular motor to move the polymer relative to the agent, and detecting signals resulting from an interaction between the units of the polymer and the agent.

The method is a method for linear analysis, in which the signals are detected sequentially. As used herein signals are detected “sequentially” when signals from different units of a single polymer are detected spaced apart in time. Not all units need to be detected or need to generate a signal to detect signals “sequentially.” When the units are sequentially exposed to the agent or station the unit and the agent or station move relative to one another. As used herein the phrase “the unit and the agent move relative to one another” means that either the unit and the agent are both moving or only one of the two is moving and the other remains stationary at least during the period of time of the interaction between the unit and the agent.

The unit and the agent are moved relative to one another by a molecular motor. A “molecular motor” as used herein is a biological molecule which physically interacts with a polymer and moves the polymer past a signal station. Preferably the molecular motor is a molecule such as a protein or protein complex that interacts with a polymer and moves with respect to the polymer along the length of the polymer. The molecular motor interacts with each unit of the polymer in a sequential manner. The physical interaction between the molecular motor and the polymer is based on molecular forces occurring between molecules such as, for instance, van der Waals forces. The type of molecular motor useful according to the methods of the invention depends on the type of polymer being analyzed. For instance a molecular motor such as e.g., a DNA polymerase or a helicase is useful when the polymer is DNA, a molecular motor such as RNA polymerase is useful when the polymer is RNA, and a molecular motor such as myosin is useful for example when the polymer is a peptide such as actin. Molecular motors include, but are not limited to, helicases, RNA polymerases, DNA polymerases, kinesin, dynein, actin, and myosin. Those of ordinary skill in the art would easily be able to identify other molecular motors useful according to the invention, based on the parameters described herein.

DNA polymerases have been demonstrated to function as efficient molecular motors. Preferably the internal diameters of the regions of the polymerase which clamp onto the DNA is similar to that of double stranded DNA. Large amounts of DNA can be threaded through the clamp in a linear fashion. The overall structure of the b-subunit of DNA polymerase III holoenzyme is 80 angstroms diameter with an internal diameter of about 35 angstroms. In comparison, a full turn of duplex B-form DNA is about 34 angstroms. The beta subunit fits around the DNA, in a mechanism referred to as a sliding clamp mechanism, to mediate the processive motion of the holoenzyme during DNA replication. It is well understood that the b-subunit encircles DNA during replication to confer processivity to the holoenzyme (Bloom et al., J. Biol. Chem., 271:30699-708, 1996; Fu et al., EMBO J., 15:4414-22, 1996; Griep, Anal. Biochem., 232:180-9, 1995; Herendeen and Kelly, Cell, 84:5-8, 1996; Naktinis et al., Cell, 84(1):137-145, 1996; Paz-Elizur et al., J. Biol. Chem., 271:2482-90, 1996 and Skaliter et al., J. Biol. Chem., 271:2491-6, 1996). Because the sliding clamp is the mechanism of processivity for a polymerase, it necessarily means that large amounts of DNA are threaded through the clamp in a linear fashion. Several kilobases are threaded through the clamp at one time (Kornberg and Baker, DNA Replication, W. H. Freeman, New York, 1991).

RNA polymerases, like DNA polymerases, can also function as efficient molecular motors. The internal diameter of the region of the RNA polymerase is such that it is capable of clamping onto the RNA and moving down the RNA in a unit by unit progression. RNA polymerases include, for instance, T7 RNA polymerase, T3 or SP6 RNA polymerases, E. coli RNA polymerases, and the like. Suitable conditions for RNA transcription using RNA polymerases are known in the art.

Another preferred type of molecular motor is a helicase. Helicases have previously been described, e.g., see U.S. Pat. No. 5,888,792. Helicases are proteins which move along nucleic acid backbones and unwind the nucleic acid so that the processes of DNA replication, repair, recombination, transcription, mRNA splicing, translation and ribosomal assembly can take place. Helicases include both RNA and DNA helicases. Nucleic acid molecular motors include those molecular motors that move along the backbone of a nucleic acid molecule and include, for instance, polymerases and helicases.

Multiple polymers can be analyzed simultaneously by causing more than one polymer to move relative to respective signal stations on respective molecular motors. The polymers may be similar or distinct. If the polymers are similar, the same or different units may be detected simultaneously. The movement of the polymer may be accomplished by the molecular motor alone or may be assisted by the use of a channel, groove or ring to guide the polymer. Alternatively the molecular motor and agent may be moved and the polymer may remain stationary. For instance the agent may be attached to the molecular motor and the polymer may be secured to a surface. In this case the molecular motor with the agent attached can scan down the length of the stationary polymer.

The method of the invention is described with respect to the following non-limiting example, which is provided for illustrative purposes only. The example refers to the analysis of DNA and fluorescence, but those of ordinary skill in the art would understand that it is applicable to all polymers and all claimed detection systems. In the example, a DNA polymerase is labeled with several fluorescent molecules, e.g. donor fluorescent molecules. A DNA molecule labeled with a matching fluorophore, e.g. an acceptor fluorophore, is then used as a template for the DNA polymerase which begins to undergo primer extension. As the acceptor fluorophore moves past the donor fluorophore, fluorescence resonance energy transfer (FRET) occurs. FRET occurs when the donor and acceptor fluorophores undergo a close range interaction in the range of approximately 1 angstrom to 100 angstroms. This distance is achieved when a single nucleotide with a label passes the fluorophore on the polymerase.

FRET analysis using molecular motors can be performed on single molecules in solution or as parallel reactions on a solid planer medium. It may also be performed in parallel reactions in different solutions such as in multi-well dishes. In the embodiment in which the reaction is carried out on a planer solid medium, either the labeled polymer or the labeled molecular motor may be immobilized directly or through a linker onto the surface. If the polymer is attached to the surface, then molecular motor can be added subsequently and if the molecular motor is tethered to the surface, then the polymer may be added to initiate the reaction. In this manner, simultaneous linear reading of multiple donor-acceptor reaction sites can occur to enhance the throughput of the system. When the molecular motor is a DNA polymerase, the sequence of several kilobases of DNA can be obtained rapidly. The approximate rate of sequencing can approach 1 megabase/hour with a 1 camera system.

The preparation of fluorescently labeled enzyme and protein complexes which can serve as molecular motors, is well known in the art. The availability of multiple amine, carboxyl, and sulfhydryl sites on enzymes makes conjugation of labels to these molecules straightforward. Many proteins have been functionalized to produce fluorescent derivatives without loss of activity, including, for instance, antibodies, horseradish peroxidase, glucose oxidase, b-galactosidase, alkaline phosphatase, actin, and myosin. Molecular motors can be easily derivatized in a similar manner, without losing functional activity. Additionally, labels can be incorporated into the polymer using methods known in the art, such as those described in U.S. Pat. No. 6,355,420. For instance, the label can be incorporated into the polymer using commercially available nucleotide or amino acid polymers or as succinimydyl ester derivatives which can be linked to primary amino groups.

Many fluorescent labels commercially available have functional groups which enable their conjugation to a protein such as a molecular motor and/or a polymer. These labels include, but are not limited to, fluorescein derivatives such as fluorescein isothiocyanate, NHS-fluorescein, iodoacetamidofluorescein, fluorescein-5-maleimide, SAMSA-fluorescein, fluorescein-5-thiosemicarbazide, and others. One of ordinary skill in the art has great flexibility in choosing a label for the methods of the invention because of the wide selection of conjugation techniques available for even just one type of label. Variants of rhodamines, Cy-dyes (Amersham-Pharmacia), Alexa dyes (Molecular probes), Texas Reds, and others make for a wide range of available wavelengths, photostabilities, energy transfer spectrum, and chemical compatibility. Good absorption, stable excitation, and efficient, high fluorescence quantum yield are important characteristics of the label.

As an example of fluorescent conjugation, fluorescein isothiocyanate (FITC) conjugation to a molecular motor is described. Fluorescein isothiocyanate is a prototypical fluorescent dye. It exists in two structural isomers, one modified in the lower ring at the 5-position or the 6-position. The two isoforms are optically equivalent in terms of fluorescent properties. The isothiocyanate group reacts with nucleophiles such as amines and sulfhydryls, however the only stable product is with primary amine groups such as the E- and N-terminal amines in proteins. The reaction between the isothiocyanate group and FITC yields a thiourea linkage and no leaving group. FITC is dissolved in DMF as a stock solution and then added to the aqueous reaction mixture at a pH above 6. Storage is at −20° C., protected from light, and under desiccated conditions. Absorbance maximum of FITC is at 495 μm and the emission maximum is at 520 nm. The solution of enzyme is usually prepared in 0.1M sodium carbonate, pH 9, and at a concentration of at least 2 mg/ml. The FITC is dissolved to a stock in DMSO/DMF at a concentration of 1 mg/ml and protected from light. In a darkened laboratory, 50-100 μl of the FITC solution is added to each milliliter of protein solution (assuming 2 mg/ml). The reaction is overnight at 4° C. The reaction is stopped by the addition of ammonium chloride to a final concentration of 50 mM. The remaining isothiocyanate groups are blocked after two additional hours. The derivative is purified using gel filtration with a PBS buffer.

In a preferred embodiment the fluorescent dye and its energy transfer pair is carefully selected to maximize signal production. This can be accomplished by considering the parameters described by the formula set forth below. Fluorescence energy transfer (FRET) directly related to the spectral overlap of the donor fluorescence emission and the acceptor fluorescence absorbance is determined as J, the normalized spectral overlap of the donor emission (fD) acceptor absorption (ε_{A}). The equation which summarizes the importance of the normalized spectral overlap is given as:

J=∫εA(λ)fD(λ)λ⁴dλ/∫fD(λ)dλ.

The J factor is especially important in the determination of the Forster energy transfer distance which is the distance at which energy transfer from donor fluorophore to acceptor fluorophore is 50%. The Forster distance also determines the resolution of the FRET sequencing method. In general the Forster distance can be varied to be between as small as 5 angstroms and 100 angstroms.

These variables have been considered in the choice of the optimal donor-acceptor pair for use in our FRET sequencing system. The J factor is important, but there are additional factors which should be worked into the system for optimal performance such as 1) the sharpness of the spectral bands, 2) the lack of crosstalk between the spectral bands, 3) the ability to immobilize the chosen labels in a polymeric matrix, and 4) the ability to have a match with common labels used for incorporation into DNA.

Other factors can be considered in choosing the proper fluorescent label pair. For instance, the spectral overlap of the labels should be sufficient for energy transfer. By minimalizing direct excitation of the acceptor fluorophore crosstalk in excitation levels can be avoided. Additionally, the emission of the donor fluorophore should not interfere with the detection band from the acceptor fluorophore. In this manner, the measured fluorescent events will be suitable and indicative of the occurrence of energy transfer. Under ideal conditions, the donor and acceptor fluorescence is sharp and not subject to spectral broadening. Furthermore, there are considerations in the quantum yield, photostability, and cross-sectional areas of the labels. All of these parameters can easily be manipulated by one of skill in the art based on the known properties of known and commercially available labels.

Those of ordinary skill in the art can verify the extent of fluorescent labeling of the molecular motor and/or polymer. The level of fluorescence labeling in the fluorophore conjugated molecule is determined by either the absorbance or the fluorescence emission of the sample. The number of fluorophore molecules per molecule is called the F/M ratio. This value is measured for all preparations of enzyme-fluorophore complexes. The ideal F/M ratio is determined for the particular molecule (molecular motor or polymer) molecule-fluorophore combination. Using the known extinction coefficient of the fluorophore, a determination of the derivitization level can be made after excess of the fluorophore is removed.

The activity of the labeled molecular motors can be verified using standard assays which assess the viability of the molecular motor fluorophore complex after conjugation and purification. Various molecular motors have their own assays for activity verification. DNA polymerase and its activity after conjugation to FITC is discussed below to clarify further on this subject. This example is in no way limiting of the scope of the invention.

DNA polymerase-fluorophore complexes are checked in dideoxy sequencing reactions to verify the ability of the modified molecular motor to perform its chain extension function. Primer annealing, labeling, and termination reactions are executed to determine the length of single-stranded, dideoxy terminated products and also to assay the base accuracy of the extended products. The reaction mixtures for the four dideoxynucleotides are subjected to four color automated capillary gel electrophoresis (such as the ABI 3770) for the final analysis. Match of the sequences with the known M13 ssDNA sequencing template confirms the integrity of the polymerase-fluorophore complexes.

In one example, an array of molecular motors (i.e. DNA polymerases) can be bound to the surface of a glass slide. The polymerases are labeled with donor fluorescent molecules which have emission spectra which partially overlap the excitation spectra of the acceptor molecule. Template acceptor labeled polymer (i.e. DNA) is provided in the reaction mixture along with the appropriate extension primers. The reaction is initiated with a mixture of deoxynucleotides. The chain extension allows the acceptor on the template DNA to be moved in proximity to the donors on the polymerase. Once the acceptor comes within energy transfer proximity to the donor on the immobilized polymerase molecule, non-radiative energy occurs. Sensitized fluorescence emission from the acceptor is induced. The temporally spaced fluorescence emission from the substrates allows for interrogation of the nucleotide information about the template molecule.

Statistical analysis of the different spatially oriented molecules allow for complete and accurate reconstruction of the sequence with speed and cost-effectiveness as discussed below in more detail. The methods of the invention thus allow for much longer read lengths and the complete elimination of separation methods, required by traditional non-linear sequencing method such as Sanger sequencing.

In another example of the linear analysis method of the invention, the template may be fixed to the glass surface and the polymerase mobile in solution. The donor fluorescence molecule may be located on the DNA molecule as opposed to the acceptor. The series of interactions may be mediated by a different molecular motor such as a helicase molecule which unwinds duplex DNA. In this scenario, the helicase molecule is fluorescently tagged and allowed to unwind complexes which are asymmetrically labeled with the fluorescent molecules. The asymmetric labeling allows for the ease of deciphering the information about the polymer.

In another example the molecular motor and polymer may be in solution. The methods of analysis can be accomplished without either the molecular motor or the polymer being attached to a surface. The molecular motor and polymer can move with respect to each other in a solution. When a single molecular motor is present in the solution, individual signals arising from the interaction can be detected and analyzed by standard methods of analysis.

The methods involved in tethering the polymer or the molecular motor to a support, labeling the system components, causing the interaction between the molecular motor and the polymer, and detection methods e.g., fluorescence resonance energy transfer etc, are described herein as well as in WO 98/35012 and U.S. Pat. No. 6,355,420. Other methods for performing labeling, immobilizing biomolecules, etc are known to those of ordinary skill in the art. For instance, Schafer, et al., Nature, 352(6334):444-8, 1991, describes methods of labeling and detection.

The invention encompasses improved methods of analyzing a polymer by detecting a signal that results from an interaction between at least one unit of the polymer and an agent or when the unit is exposed to the station. By “analyzing” a polymer, it is meant obtaining some information about the structure of the polymer such as its size, the order of its units, its relatedness to other polymers, the identity of its units, or its presence. Since the structure and function of biological molecules are interdependent, the structural information can reveal important information about the function of the polymer.

The methods of the invention also are useful for identifying other structural properties of polymers. The structural information obtained by analyzing a polymer according to the methods of the invention may include the identification of characteristic properties of the polymer which (in turn) allows, for example, for the identification of the presence of a polymer in a sample or a determination of the relatedness of polymers, identification of the size of the polymer, identification of the proximity or distance between two or more individual units of a polymer, identification of the order of two or more individual units within a polymer, and/or identification of the general composition of the units of the polymer. Such characteristics are useful for a variety of purposes such as determining the presence or absence of a particular polymer in a sample. For instance when the polymer is a nucleic acid the methods of the invention may be used to determine whether a particular genetic sequence is expressed in a cell or tissue. The presence or absence of a particular sequence can be established by determining whether any polymers within the sample express a characteristic pattern of individual units which is only found in the polymer of interest i.e., by comparing the detected signals to a known pattern of signals characteristic of a known polymer to determine the relatedness of the polymer being analyzed to the known polymer. The entire sequence of the polymer of interest does not need to be determined in order to establish the presence or absence of the polymer in the sample. Similarly the methods may be useful for comparing the signals detected from one polymer to a pattern of signals from another polymer to determine the relatedness of the two polymers.

The proximity of or distance between two individual units of a polymer may be determined according to the methods of the invention. It is important to be able to determine the proximity of or distance between two units for several reasons. Each unit of a polymer has a specific position along the backbone. The sequence of units serves as a blueprint for a known polymer. The distance between two or more units on an unknown polymer can be compared to the blueprint of a known polymer to determine whether they are related. Additionally the ability to determine the distance between two units is important for determining how many units, if any, are between the two units of interest.

In general the methods of linear polymer analysis of the invention are performed by detecting signals arising from an interaction between a labeled unit of the polymer and an agent selected from the group consisting of an electromagnetic radiation source, a quenching source and a fluorescence excitation source. A “signal” as used herein is a detectable physical quantity which transmits or conveys information about the structural characteristics of a labeled unit of a polymer and which is capable of being detected. Preferably the physical quantity is electromagnetic radiation. The signal may arise from energy transfer, quenching, radioactivity etc. Although the signal is specific for a particular labeled unit, a polymer having more than one of a particular labeled unit will have more than one identical signal. Additionally, each labeled unit of a specific type may give rise to different signals if they have different labels.

The method used for detecting the signal depends on the type of physical quantity generated. For instance if the physical quantity is electromagnetic radiation then the signal is optically detected. An “optically detectable” signal as used herein is a light based signal in the form of electromagnetic radiation which can be detected by light detecting imaging systems.

A “plurality of polymers” is at least two polymers. A plurality of polymers in one embodiment is at least 50 polymers and in another embodiment is at least 100 polymers.

The signals may provide any type of structural information about the polymer. For instance these signals may provide the entire or portions of the entire sequence of the polymer, the order of signals, or the time of separation between signals as an indication of the distance between the labeled units.

As used herein “similar polymers” are polymers which have at least one overlapping region. Similar polymers may be a homogeneous population of polymers or a heterogeneous population of polymers. A “homogeneous population” of polymers as used herein is a group of identical polymers. A “heterogeneous population” of similar polymers is a group of similar polymers which are not identical but which include at least one overlapping region of identical units. An overlapping region in a nucleic acid typically consists of at least 10 contiguous nucleotides. In some cases an overlapping region consists of at least 11, 12, 13, 14, 15, 16, 17, 18, 19, 20, 21, or 22 contiguous nucleotides.

A “polymer” as used herein is a compound having a linear backbone of individual units which are linked together by linkages. In some cases the backbone of the polymer may be branched. Preferably the backbone is unbranched. The term “backbone” is given its usual meaning in the field of polymer chemistry. The polymers may be heterogeneous in backbone composition thereby containing any possible combination of polymer units linked together such as peptide-nucleic acids (which have amino acids linked to nucleic acids and have enhanced stability). In a preferred embodiment the polymers are homogeneous in backbone composition and are, for example, nucleic acids, polypeptides, polysaccharides, carbohydrates, polyurethanes, polycarbonates, polyureas, polyethyleneimines, polyarylene sulfides, polysiloxanes, polyimides, polyacetates, polyamides, polyesters, or polythioesters. In the most preferred embodiments, the polymer is a nucleic acid or a polypeptide. A “nucleic acid” as used herein is a biopolymer comprised of nucleotides, such as deoxyribose nucleic acid (DNA) or ribose nucleic acid (RNA). A polypeptide as used herein is a biopolymer comprised of linked amino acids.

As used herein with respect to linked units of a polymer, “linked” or “linkage” means two entities are bound to one another by any physicochemical means. Any linkage known to those of ordinary skill in the art, covalent or non-covalent, is embraced. Such linkages are well known to those of ordinary skill in the art. Natural linkages, which are those ordinarily found in nature connecting the individual units of a particular polymer, are most common. Natural linkages include, for instance, amide, ester and thioester linkages. The individual units of a polymer analyzed by the methods of the invention may be linked, however, by synthetic or modified linkages. Polymers where the units are linked by covalent bonds will be most common but also include hydrogen bonded, etc.

The polymer is made up of a plurality of individual units. An “individual unit” as used herein is a building block or monomer which can be linked directly or indirectly to other building blocks or monomers to form a polymer. The polymer preferably is a polymer of at least two different linked units. The at least two different linked units may produce or be labeled to produce different signals, as discussed in greater detail below. The particular type of unit will depend on the type of polymer. For instance DNA is a biopolymer composed of a deoxyribose phosphate backbone composed of units of purines and pyrimidines such as adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. RNA is a biopolymer comprised of a ribose phosphate backbone composed of units of purines and pyrimidines such as those described for DNA but wherein uracil is substituted for thymidine. The DNA nucleotides may be linked to one another by their 5′ or 3′ hydroxyl group thereby forming an ester linkage. The RNA nucleotides may be linked to one another by their 5′, 3′ or 2′ hydroxyl group thereby forming an ester linkage. Alternatively, DNA or RNA units having a terminal 5′, 3′ or 2′ amino group may be linked to the other units of the polymer by the amino group thereby forming an amide linkage.

Whenever a nucleic acid is represented by a sequence of letters it will be understood that the nucleotides are in 5′→3′ order from left to right and that “A” denotes adenosine, “C” denotes cytidine, “G” denotes guanosine, “T” denotes thymidine, and “U” denotes uracil unless otherwise noted.

The polymers may be native or naturally-occurring polymers which occur in nature or non-naturally occurring polymers which do not exist in nature. The polymers typically include at least a portion of a naturally occurring polymer. The polymers can be isolated or synthesized de novo. For example, the polymers can be isolated from natural sources e.g. purified, as by cleavage and gel separation or may be synthesized e.g.,(i) amplified in vitro by, for example, polymerase chain reaction (PCR); (ii) synthesized by, for example, chemical synthesis; (iii) recombinantly produced by cloning, etc.

The polymer or at least one labeled unit thereof is in a form which is capable of interacting with an agent or station to produce a signal characteristic of that interaction. The labeled unit of a polymer which is capable of undergoing such an interaction is said to be labeled. If a labeled unit of a polymer can undergo that interaction to produce a characteristic signal, then the polymer is said to be intrinsically labeled. It is not necessary that an extrinsic label be added to the polymer. If a non-native molecule, however, must be attached to the individual labeled unit of the polymer to generate the interaction producing the characteristic signal, then the polymer is said to be extrinsically labeled. The “label” may be, for example, light emitting, energy accepting, fluorescent, radioactive, or quenching. In some embodiments the labeled polymer is an extrinsically labeled polymer and in other embodiments, it is an intrinsically labeled polymer.

Many naturally occurring units of a polymer are light emitting compounds or quenchers. For instance, nucleotides of native nucleic acid molecules have distinct absorption spectra, e.g., A, G, T, C, and U have absorption maximums at 259 nm, 252 nm, 267 um, 271 nm, and 258 nm respectively. Modified units which include intrinsic labels may also be incorporated into polymers. A nucleic acid molecule may include, for example, any of the following modified nucleotide units which have the characteristic energy emission patterns of a light emitting compound or a quenching compound: 2,4-dithiouracil, 2,4-diselenouracil, hypoxanthine, mercaptopurine, 2-aminopurine, and selenopurine.

The types of labels useful according to the methods of the invention, guidelines for selecting the appropriate labels, and methods for adding extrinsic labels to polymers are provided in more detail in WO 98/35012 and U.S. Pat. No. 6,355,420.

A “labeled unit” as used herein is any labeled unit in a polymer that identifies a particular unit or units. A labeled unit includes, for instance, fluorescent markers and intrinsically and extrinsically labeled units.

A method for characterizing a test polymer is performed by obtaining polymer dependent impulses for each of a plurality of polymers, comparing the polymer dependent impulses of the plurality of polymers, determining the relatedness of the polymers based upon similarities between the polymer dependent impulses of the polymers, and characterizing the test polymer based upon the polymer dependent impulses of related polymers.

A “polymer dependent impulse” as used herein is a detectable physical quantity which transmits or conveys information about the structural characteristics of only a single unit of a polymer. The physical quantity may be in any form which is capable of being detected. For instance the physical quantity may be electromagnetic radiation, chemical conductance, electrical conductance, etc. The polymer dependent impulse may arise from energy transfer, quenching, changes in conductance, mechanical changes, resistance changes, or any other physical changes. Although the polymer dependent impulse is specific for a particular unit, a polymer having more than one of a particular labeled unit will have more than one identical polymer dependent impulse. Additionally, each unit of a specific type may give rise to different polymer dependent impulses if they have different labels.

The method used for detecting the polymer dependent impulse depends on the type of physical quantity generated. For instance if the physical quantity is electromagnetic radiation then the polymer dependent impulse is optically detected. An “optically detectable” polymer dependent impulse as used herein is a light based signal in the form of electromagnetic radiation which can be detected by light detecting imaging systems. When the physical quantity is chemical conductance then the polymer dependent impulse is chemically detected. A “chemically detected” polymer dependent impulse is a signal in the form of a change in chemical concentration or charge such as an ion conductance which can be detected by standard means for measuring chemical conductance. If the physical quantity is an electrical signal then the polymer dependent impulse is in the form of a change in resistance or capacitance.

As used herein the “relatedness of polymers” can be determined by identifying a characteristic pattern of a polymer which is unique to that polymer. For instance if the polymer is a nucleic acid then virtually any sequence of 10 contiguous nucleotides within the polymer would be a unique characteristic of that nucleic acid molecule. Any other nucleic acid molecule which displayed an identical sequence of 10 nucleotides would be a related polymer.

A “plurality of polymers” is at least two polymers. Preferably a plurality of polymers is at least 50 polymers and more preferably at least 100 polymers.

The polymer dependent impulses may provide any type of structural information about the polymer. For instance these signals may provide the entire or portions of the entire sequence of the polymer, the order of polymer dependent impulses, or the time of separation between polymer dependent impulses as an indication of the distance between the units.

The polymer dependent impulses are obtained by interaction which occurs between the unit of the polymer and the environment at a signal generation station. A “signal generation station” as used herein is a station that is an area where the unit interacts with the environment to generate a polymer dependent impulse. In some aspects of the invention the polymer dependent impulse results from contact in a defined area with an agent selected from the group consisting of electromagnetic radiation, a quenching source, and a fluorescence excitation source which can interact with the unit to produce a detectable signal. In other aspects the polymer dependent impulse results from contact in a defined area with a chemical environment which is capable of undergoing specific changes in conductance in response to an interaction with a molecule. As a molecule with a specific structure interacts with the chemical environment a change in conductance occurs. The change which is specific for the particular structure may be a temporal change, e.g., the length of time required for the conductance to change may be indicative that the interaction involves a specific structure or a physical change. For instance, the change in intensity of the interaction may be indicative of an interaction with a specific structure. In other aspects the polymer dependent impulse results from changes in capacitance or resistance caused by the movement of the unit between microelectrodes or nanoelectrodes positioned adjacent to the polymer unit. For instance the signal generation station may include microelectrodes or nanoelectrodes positioned on opposite sides of the polymer unit. The changes in resistance or conductance which occur as a result of the movement of the unit past the electrodes will be specific for the particular unit.

A method for determining the distance between two individual units is also encompassed by the invention. In order to determine the distance between two individual units of a polymer of linked units the polymer is caused to pass linearly relative to an signal generation station and a polymer dependent impulse which is generated as each of the two individual units passes by the signal generation station is measured. Each of the steps is then repeated for a plurality of similar polymers. A polymer is said to pass linearly relative to a signal generation station when each unit of the polymer passes sequentially by the signal generation station.

Each of the steps is repeated for a plurality of similar polymers to produce a data set. The distance between the two individual units can then be determined based upon the information obtained from the plurality of similar polymers by analyzing the data set.

The method also includes a method for identifying a quantity of polymers including a label. For instance, it is possible to determine the number of polymers having a specific unit or combination of units in a sample. In a sample of MRNA, for example, the number of a particular mRNA present in the sample can be determined. This is accomplished by identifying a pattern or signature characteristic of the desired mRNA molecule. The sample of RNA can then be analyzed according to the methods of the invention and the number of mRNA molecules having the specific pattern or signature can be determined.

Currently, less than 5% of the human genome has been sequenced. This translates into a small fraction of the ideal in human sequence knowledge, which is the sequence of all individuals. For instance, for the human population, there are 1.4×10¹⁹ (5 billion people×3.×10⁹ bases/person). So far, only 2×10⁻¹⁰ percent of all human genetic information is known. The rate of sequencing of the human genome by all world-wide efforts is roughly 3×10⁹/15 years, or 550,000 bases/day, at a cost of >$1/base. Sequencing by the methods of the invention described herein will constitute an inordinate breakthrough in the rate of sequencing. The predicted time to complete one human genome with one machine is about 15 hours. Several dynamic arrays in parallel will be able to complete the sequence of one human genome in a fraction of an hour.

A method for sequencing a polymer of linked units is also encompassed by the invention. The method is performed by obtaining polymer dependent impulses from each of a plurality of overlapping polymers, at least a portion of each of the polymers having a sequence of linked units identical to the other of the polymers, and comparing the polymer dependent impulses to obtain a sequence of linked units which is identical in the plurality of polymers.

The plurality of overlapping polymers is a set of polymers in which each polymer has at least a portion of its sequence of linked units which is identical to the other polymers. The portion of sequence which is identical is referred to as the overlapping region and which includes at least ten contiguous units.

In another aspect of the invention the order of units of a polymer of linked units can be determined by moving the polymer linearly relative to a signal generation station and measuring a polymer dependent impulse generated as each of two individual units, each giving rise to a characteristic polymer dependent impulse pass by the signal generation station. These steps are repeated for a plurality of similar polymers and the order of at least the two individual units is determined based upon the information obtained from the plurality of similar polymers.

A method for analyzing a set of polymers, in which each of the polymers of the set is an individual polymer of linked units, is encompassed by the invention. The method involves the step of orienting the set of polymers parallel to one another, and detecting a polymer specific feature of the polymers.

The set of polymers are oriented parallel to one another. The polymers may be oriented by any means which is capable of causing the polymers to be positioned parallel to one another. For instance an electric field may be applied to the polymers to cause them to be oriented in a parallel form. Preferably the orientation step is in a solution free of gel.

A “polymer specific feature” as used herein is any structural feature of polymer which relates to its sequence. For instance a polymer specific feature includes but is not limited to information about the polymer such as the length of the polymer, the order of linked units in the polymer, the distance between units of the polymer, the proximity of units in the polymer, the sequence of one, some or all of the units of the polymer, and the presence of the polymer.

By including more than one physical characteristic into the label, the simultaneous and overlapping reading of the nucleic acid within the same temporal frame may provide more accurate and rapid information about the positions of the labeled nucleotides than when only a single physical characteristic is included. The sample may be, for instance, labeled with different wavelength fluorophores. Each of the fluorophores can be detected separately to provide distinct readings from the same sample. For instance, the end units of a polymer may be labeled with fluorophores which emit at a first wavelength and a set of internal units may be labeled with a fluorophore which emits at a second wavelength. As the polymer is moved past the signal station both wavelengths can be detected to provide information about both sets of labels.

One use for the methods of the invention is to determine the sequence of units within a polymer. Identifying the sequence of units of a polymer, such as a nucleic acid, is an important step in understanding the function of the polymer and determining the role of the polymer in a physiological environment such as a cell or tissue. The sequencing methods currently in use are slow and cumbersome. The methods of the invention are much quicker and generate significantly more sequence data in a very short period of time.

The detectable signal is produced at a signal station. A “signal station” as used herein is a region where a portion of the polymer to be detected, e.g. the labeled unit, is exposed to, in order to produce a signal or signal. The station may be composed of any material including a gas. Preferably the station is a non-liquid material. “Non-liquid” has its ordinary meaning in the art. A liquid is a non-solid, non-gaseous material characterized by free movement of its constituent molecules among themselves but without the tendency to separate. In another preferred embodiment the station is a solid material.

The signal station is an interaction station. As used herein an “interaction station or site” is a region where a labeled unit of the polymer interacts with an agent and is positioned with respect to the agent in interactive proximity. “Interactive proximity” as used herein means that the unit and the agent are in close enough proximity whereby they can interact. The interaction station for fluorophores, for example, is that region where they are close enough so that they energetically interact to produce a signal.

The interaction station in a preferred embodiment is a region of a molecular motor where a localized agent, such as an acceptor fluorophore, attached to the molecular motor or support can interact with a polymer passing through the molecular motor. The point where the polymer passes the localized region of agent is the interaction station. As each labeled unit of the polymer passes by the agent a detectable signal is generated. The agent may be localized within the region of the channel in a variety of ways. For instance the agent may be physically attached to the molecular motor, directly or by a linker, at the site where the polymer interacts with the molecular motor. Alternatively, the molecular motor may be attached to a support and the agent may also be attached to the support, as long as the agent is attached to a region of the support by which all units of the polymer will pass. For instance, the agent may be embedded in a material or on the surface of a material that forms the wall of a channel wherein the molecular motor is attached to the wall and moves the polymer through the channel. Alternatively the agent may be a light source which is positioned a distance from the molecular motor or support but which is capable of transporting light directly to a region of the channel through a waveguide. These and other related embodiments of the invention are discussed in more detail below. The movement of the polymer may be assisted by the use of a groove or ring to guide the polymer.

Other arrangements for creating interaction stations are embraced by the invention. For example, a polymer can be passed through a molecular motor tethered to the surface of a wall or embedded in a wall, thereby bringing labeled units of the polymer sequentially to a specific location, preferably in interactive proximity to a proximate agent, thereby defining an interaction station. A molecular motor is a compound such as polymerase, helicase, or actin which interacts with the polymer and is transported along the length of the polymer past each labeled unit. Likewise, the polymer can be held from movement and a reader can be moved along the polymer, the reader being a molecular motor and having attached to it the agent.

The agent that interacts with the labeled unit of the polymer at the interaction station is selected from the group consisting of electromagnetic radiation, a quenching source, and a fluorescence excitation source. “Electromagnetic radiation” as used herein is energy produced by electromagnetic waves. Electromagnetic radiation may be in the form of a direct light source or it may be emitted by a light emissive compound such as a donor fluorophore. “Light” as used herein includes electromagnetic energy of any wavelength including visible, infrared and ultraviolet.

As used herein, a quenching source is any entity which alters or is capable of altering a property of a light emitting source. The property which is altered can include intensity fluorescence lifetime, spectra, fluorescence, or phosphorescence.

A fluorescence excitation source as used herein is any entity capable of fluorescing or giving rise to photonic emissions (i.e. electromagnetic radiation, directed electric field, temperature, fluorescence, radiation, scintillation, physical contact, or mechanical disruption.) For instance, when the labeled unit is labeled with a radioactive compound the radioactive emission causes molecular excitation of an agent that is a scintillation layer which results in fluorescence.

When a labeled unit of the polymer is exposed to the agent the interaction between the two produces a signal. The signal provides information about the polymer. For instance if all labeled units of a particular type, e.g., all of the alanines, of a protein polymer are labeled (intrinsic or extrinsic) with a particular light emissive compound then when a signal characteristic of that light emissive compound is detected upon interaction with the agent the signal signifies that an alanine residue is present at that particular location on the polymer. If each type of labeled unit e.g., each type of amino acid is labeled with a different light emissive compound having a distinct light emissive pattern then each amino acid will interact with the agent to produce a distinct signal. By determining what each signal for each labeled unit of the polymer is, the sequence of units can be determined.

The interaction between the labeled unit and the agent can take a variety of forms, but does not require that the labeled unit and the agent physically contact one another. Examples of interactions are as follows. A first type of interaction involves the agent being electromagnetic radiation and the labeled unit of the polymer being a light emissive compound (either intrinsically or extrinsically labeled with a light emissive compound). When the light emissive labeled unit is contacted with electromagnetic radiation (such as by a laser beam of a suitable wavelength or electromagnetic radiation emitted from a donor fluorophore), the electromagnetic radiation causes the light emissive compound to emit electromagnetic radiation of a specific wavelength. The signal is then measured. The signal exhibits a characteristic pattern of light emission and thus indicates that a particular labeled unit of the polymer is present. In this case the labeled unit of the polymer is said to “detectably affect the emission of the electromagnetic radiation from the light emissive compound”.

A second type of interaction involves the agent being a fluorescence excitation source and the labeled unit of the polymer being a light emissive or a radioactive compound. When the light emissive labeled unit is contacted with the fluorescence excitation source, the fluorescence excitation source causes the light emissive compound to emit electromagnetic radiation of a specific wavelength. When the radioactive labeled unit is contacted with the fluorescence excitation source, the nuclear radiation emitted from the labeled unit causes the fluorescence excitation source to emit electromagnetic radiation of a specific wavelength. The signal then is measured.

A variation of these types of interaction involves the presence of a third element of the interaction, a proximate compound which is involved in generating the signal. For example, a labeled unit may be labeled with a light emissive compound which is a donor fluorophore and a proximate compound can be an acceptor fluorophore. If the light emissive compound is placed in an excited state and brought proximate to the acceptor fluorophore, then energy transfer will occur between the donor and acceptor, generating a signal which can be detected as a measure of the presence of the labeled unit which is light emissive. The light emissive compound can be placed in the “excited” state by exposing it to light (such as a laser beam) or by exposing it to a fluorescence excitation source.

Another interaction involves a proximate compound which is a quenching source. In this instance, the light emissive labeled unit is caused to emit electromagnetic radiation by exposing it to light. If the light emissive compound is placed in proximity to a quenching source, then the signal from the light emissive labeled unit will be altered.

A set of interactions parallel to those described above can be created wherein, however, the light emissive compound is the proximate compound and the labeled unit is either a quenching source or an acceptor source. In these instances the agent is electromagnetic radiation emitted by the proximate compound, and the signal is generated, characteristic of the interaction between the labeled unit and such radiation, by bringing the labeled unit in interactive proximity with the proximate compound.

The mechanisms by which each of these interactions produces a detectable signal is known in the art. For exemplary purposes the mechanism by which a donor and acceptor fluorophore interact according to the invention to produce a detectable signal including practical limitations which are known to result from this type of interaction and methods of reducing or eliminating such limitations is set forth below.

Another preferred method of analysis of the invention involves the use of radioactively labeled polymers. The type of radioactive emission influences the type of detection device used. In general, there are three different types of nuclear emission including alpha, beta, and gamma radiation. Alpha emission cause extensive ionization in matter and permit individual counting by ionization chambers and proportional counters, but more interestingly, alpha emission interacting with matter may also cause molecular excitation, which can result in fluorescence. The fluorescence is referred to as scintillation. Beta decay which is weaker than alpha decay can be amplified to generate an adequate signal. Gamma radiation arises from internal conversion of excitation energy. Scintillation counting of gamma rays is efficient and produces a strong signal. Sodium iodide crystals fluoresce with incident gamma radiation.

A “scintillation” layer or material as used herein is any type of material which fluoresces or emits light in response to excitation by nuclear radiation. Scintillation materials are well known in the art. Aromatic hydrocarbons which have resonance structures are excellent scintillators. Anthracene and stilbene fall into the category of such compounds. Inorganic crystals are also known to fluoresce. In order for these compounds to luminesce, the inorganic crystals must have small amounts of impurities, which create energy levels between valence and conduction bands. Excitation and de-excitation can therefore occur. In many cases, the de-excitation can occur through phosphorescent photon emission, leading to a long lifetime of detection. Some common scintillators include NaI (Ti), ZnS (Ag), anthracene, stilbene, and plastic phosphors.

Many methods of measuring nuclear radiation are known in the art and include devices such as cloud and bubble chamber devices, constant current ion chambers, pulse counters, gas counters (i.e., Geiger-Muller counters), solid state detectors (surface barrier detectors, lithium-drifted detectors, intrinsic germanium detectors), scintillation counters, Cerenkov detectors, etc.

Analysis of the radiolabeled polymers is identical to other means of generating signals. For example, a sample with radiolabeled A's can be analyzed by the system to determine relative spacing of A's on a sample DNA. The time between detection of radiation signals is characteristic of the polymer analyzed. Analysis of four populations of labeled DNA (A's, C's, G's, T's) can yield the sequence of the nucleic acid analyzed. The sequence of DNA can also be analyzed with a more complex scheme including analysis of a combination of dual labeled DNA and singly labeled DNA. Analysis of a and C labeled fragment followed by analysis of a labeled version of the same fragment yields knowledge of the positions of the A's and C's. The sequence is known if the procedure is repeated for the complementary strand. The system can further be used for analysis of polymer (polypeptide, RNA, carbohydrates, etc.), size, concentration, type, identity, presence, sequence and number.

The methods described above can be performed on a single polymer or on more than one polymer in order to determine structural information about the polymer.

A “detectable signal” as used herein is any type of electromagnetic radiation signal which can be sensed by conventional technology. The signal produced depends on the type of station as well as the labeled unit and the proximate compound if present. In one embodiment the signal is electromagnetic radiation resulting from light emission by a labeled (intrinsic or extrinsic) labeled unit of the polymer or by the proximate compound. In another embodiment the signal is fluorescence resulting from an interaction of a radioactive emission with a scintillation layer. The detected signals may be stored in a database for analysis. One method for analyzing the stored signals is by comparing the stored signals to a pattern of signals from another polymer to determine the relatedness of the two polymers. Another method for analysis of the detected signals is by comparing the detected signals to a known pattern of signals characteristic of a known polymer to determine the relatedness of the polymer being analyzed to the known polymer. Comparison of signals is discussed in more detail below.

More than one detectable signal may be detected. For instance a first individual labeled unit may interact with the agent to produce a first detectable signal and a second individual labeled unit may interact with the agent to produce a second detectable signal different from the first detectable signal. This enables more than one type of labeled unit to be detected on a single polymer.

Once the signal is generated it can then be detected. The particular type of detection means will depend on the type of signal generated which of course will depend on the type of interaction which occurs between the labeled unit and the agent. Many interactions involved in the method of the invention will produce an electromagnetic radiation signal. Many methods are known in the art for detecting electromagnetic radiation signals, including two- and three-dimensional imaging systems. These and other systems are described in more detail in PCT Application WO 98/35012 and U.S. Pat. Nos. 6,355,420 and 6,403,311.

Optical detectable signals are generated, detected and stored in a database the signals can be analyzed to determine structural information about the polymer. The computer may be the same computer used to collect data about the polymers, or may be a separate computer dedicated to data analysis. A suitable computer system to implement the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism. Computer programs for data analysis of the detected signals are readily available from CCD manufacturers.

The methods of the invention can be accomplished using any device which produces a specific detectable signal for an individual labeled unit of a polymer as the polymer moves through a molecular motor. One type of device which enables this type of analysis is one which promotes linear movement of a polymer past an interaction station using a molecular motor, wherein the interaction station includes an agent selected from the group consisting of an electromagnetic radiation source, a quenching source, a luminescent film layer, and a fluorescence excitation source. Preferably the agent is close enough to the molecular motor and is present in an amount sufficient to detectably interact with a partner compound selected from the group consisting of a light emissive compound and a quencher being moved by the molecular motor.

Preferably the molecular motor is tethered to a support. A “support” as used herein is any solid surface, such as a slide or bead, but does not include semi-solid materials such as gels or lipid bilayers.

In another preferred embodiment, neither the molecular motor or the polymer is tethered to a support. The entire method may be performed in solution, as described above.

In another embodiment the molecular motor may be tethered to a wall material having at least one channel. This arrangement is useful for guiding the polymer as it is moved by the molecular motor. A wall material is a solid or semi-solid barrier of any dimension which is capable of supporting at least one channel. A semi-solid material is a self supporting material and may be for instance a gel material such as a polyacrylamide gel. For instance the wall material may be composed of a single support material which may be conducting or non-conducting, light permeable or light impermeable, clear or unclear. In some instances the agent is embedded within the wall material. In these instances the wall material can be solely or partially made of a non-conducting layer, a light permeable layer or a clear layer to allow the agent to be exposed to the channel formed in the wall material to allow signal generation. When the wall material is only partially made from these materials the remaining wall material may be made from a conducting, light impermeable or unclear layer, which prevent signal generation. In some cases the wall material is made up of layers of different materials. For instance, the wall material may be made of a single conducting layer and a single non-conducting layer. Alternatively the wall material may be made of a single non-conducting layer surrounded by two conducing layers. Multiple layers and various combinations of materials are encompassed by the wall material of the invention.

The agent may be tethered to the wall material in this embodiment or it may be tethered to the molecular motor.

As used herein a “luminescent film layer” is a film which is naturally luminescent or made luminescent by some means of excitation or illumination, e.g., electrooptic thin films and high index films illuminated by internal reflection.

As used herein a “material shield” is any material which prevents or limits energy transfer or quenching. Such materials include but are not limited to conductive materials, high index materials, and light impermeable materials. In a preferred embodiment the material shield is a conductive material shield. As used herein a “conductive material shield” is a material which is at least conductive enough to prevent energy transfer between donor and acceptor sources.

A “conductive material” as used herein is a material which is at least conductive enough to prevent energy transfer between a donor and an acceptor.

A “nonconductive material” as used herein is a material which conducts less than that amount that would allow energy transfer between a donor and an acceptor.

A “light permeable material” as used herein is a material which is permeable to light of a wavelength produced by the specific electromagnetic radiation, quenching source, or the fluorescence excitation source being used.

A “light impermeable material” as used herein is a material which is impermeable to light of a wavelength produced by the specific electromagnetic radiation, quenching source, or the fluorescence excitation source being used.

A “channel” as used herein is a passageway through a medium through which a polymer can pass. The channel can have any dimensions as long as a polymer is capable of passing through it. For instance the channel may be an unbranched straight cylindrical channel or it may be a branched network of interconnected winding channels. Preferably the channel is a straight nanochannel or a microchannel. A “nanochannel” as used herein is a channel having dimensions on the order of nanometers. The average diameter of a nanochannel is between 1 nm and 999 nm. A “microchannel” as used herein is a channel having dimensions on the order of micrometers. The average diameter of a microchannel is between 1 mm and 1 mm. Preferred specifications and dimensions of channels useful according to the invention are set forth in detail below. In a preferred embodiment, the channel is fixed in the wall.

An agent is attached to the wall material or the molecular motor in such a manner that it will detectably interact with a partner compound by undergoing energy transfer or quenching with the partner light emissive compound which is passing through the channel of the wall material and the molecular motor. In order to interact with the partner compound the agent can be positioned in close proximity to the channel. For example, the agent may be attached to the inside of the channel, attached to the external surface of the wall material, attached to a concentrated region of the external surface of the wall material surrounding the rim of the channel, embedded within the wall material, embedded in the form of a concentric ring in the wall material surrounding the channel, attached to a localized region of the molecular motor or attached on the surface of the molecular motor. Optionally the agent may cover the entire surface of the wall material or molecular motor or may be embedded throughout. In order to improve signal generation when the agent is not localized, a mask may be used to cover some areas of the wall material or molecular motor such that only localized regions of agent are exposed. A “mask” as used herein is an object which has openings of any size or shape. More than one agent may be attached to the wall material or motor in order to produce different signals when the agents are exposed to the partner agent.

The agent may be attached to the surface of the wall material or molecular motor by any means of performing attachment known in the art. Examples of methods for conjugating biomaterials are presented in Hermanson, G. T., Bioconjugate Techniques, Academic Press, Inc., San Diego, 1996.

When the agent is attached to the surface of the wall material or molecular motor, it may be attached directly to the wall material or molecular motor or it may be attached via a linker. A “linker” as used herein with respect to the attachment of the agent is a molecule that tethers a light emitting compound or a quenching compound to the wall material or molecular motor. Linkers are well known in the art. They include hetero and homo bifunctional linkers. Commonly used linkers include alkanes of various lengths.

The agent is attached to the wall material or molecular motor in an amount sufficient to detectably interact with a partner light emissive compound. As used herein a “partner light emissive compound” is a light emissive compound as defined above but which specifically interacts with and undergoes energy transfer or quenching when positioned in close proximity to the agent. The amount of partner light emissive compound and the amount of agent required will depend on the type of agent and light emissive compound used.

As used herein a “plurality of stations” is at least two stations. Preferably a plurality of stations is at least three stations. In another preferred embodiment a plurality of stations is at least five stations.

PCT Application WO 98/35012 and U.S. Pat. No. 6,355,420 provide a detailed description of an optimal design of a nanochannel plate having fluorophores embedded within the plate as well as other articles useful for practicing the methods of the invention. The methods of the invention are not limited, however, to the use of articles of manufacture described herein or in the priority PCT application. The examples are provided for illustrative purposes only. The methods of the invention can be performed using any system in which a plurality of labeled units of a polymer can be moved with respect to a fixed station and from which signals can be obtained.

Each of the above described nanochannels useful with the molecular motor is only an example. It is, therefore, anticipated that each of the limitations described with respect to these embodiments involving any one element or combinations of elements can be included in each nanochannel. Preparation of films having multiple layers of differing material have been described in the art, e.g., U.S. Pat. No. 5,462,467, Ferreira et. al., Thin Solid Films, 244:806-809, 1994.

### EXAMPLES

### Example 1

### Method of Determining Genetic Locus for Eye Color

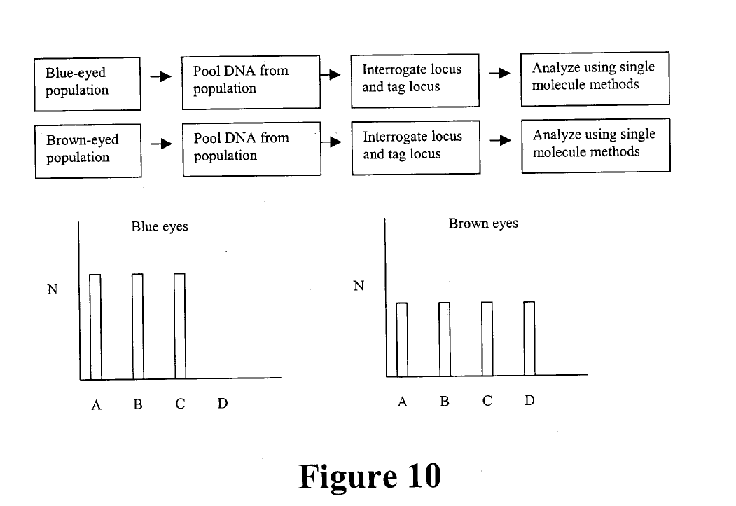

The general schematic of DNA pooling for population analysis using single molecule genetic analysis is shown in FIG. 10. As a concrete example, the DNA from the populations are pooled, the locus is amplified using the polymerase chain reaction (PCR), tagged fluorescently for haplotypes, cleaned, and introduced into the single molecule analyzer/nanochip configuration. A brief description of the schema used is described for haplotype analysis using four-color single molecule analysis of four primer extended bases. In this particular embodiment, the primer extension reaction is used with four differently labeled dNTPs. Each dNTP has a different spectrally distinguishable fluorophore. In this particular case if the coincident detection of the various color combinations are analyzed.

In this scenario, with a pair of SNPs that allow for four-color spectral discrimination, the use of four color analysis facilitates unambiguous discrimination of the four bases. In this manner, each of the four haplotypes are determined by a unique color combination. The resulting data from the analysis allows the determination of the presence or absence of particular haplotypes in the population. The PCR analysis amplifies all of the DNA from the population and the ability to count the individual haplotypes results in the determination of which haplotypes are present in the population and where there may be differences. Without single molecule analysis, the tagging and pooling schema would not allow for proper determination of the haplotypes. If the bulk fluorescence from the pooled DNA were measured, then in each of the populations, the four colors used for the analysis would be present and individual haplotypes would not be able to be determined. A sample data output for this example is also shown in FIG. 10.

From the data output of the pooled populations of DNA, one can determine the causative haplotype for the phenotype in question. In this case, the difference in presence or absence of haplotypes between the two populations is haplotype D. For eye color, this haplotype D recognizes a dominant allele that, if present, would confer the brown eye color. This pooling method is thus extremely powerful and allows the correlation of populations using a simple pooling, reaction, and analysis procedure.

### Example 2

### Pooling: Long PCR, Tagging, Linear Analysis

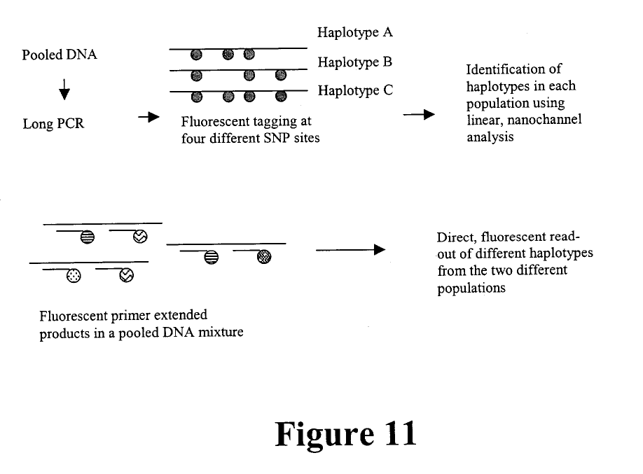

The DNA from case and control are pooled respectively. The two reaction mixtures are then amplified using long PCR to generate reaction products that are 15 kilobases long. Each of the reaction products are then tagged at four different SNPs along the length of the PCR product (FIG. 11). The tagging is accomplished using fluorescently-tagged oligonucleotides. The oligonucleotides are targeted towards the SNPs of interest and tagged with the same color fluorophore. The PCR product is then introduced into a nanochannel system. In the nanochannel system, the fluorescently-tagged DNA is introduced into the nanochannel system and driven hydrodynamically through small nanometer-sized channels. The constriction of the channels allows the DNA to be elongated and read in a linear fashion. The linear analysis thus allows the determination of haplotypes present in the population of molecules. The molecular signature that arises from each of the molecules passing through the nanochannel system represents one particular haplotype in the population. The following diagram schematically illustrates the process of haplotype analysis using pooling, PCR, and linear analysis.

### Example 3

### Pooling: Sequence-Specific Fluorescent Tagging, Single Molecule Analysis

In this particular example, the DNA from the populations is pooled and the DNA is directly tagged fluorescently without prior amplification and then introduced into the reaction chamber for single molecule analysis (FIG. 11). The DNA in this case is tagged at two different SNP sites using primer extension of sequence-specific primers. At each of the sites that are interrogated, there are four possible haplotypes with the different combinations. The presence or absence of a particular haplotype correlated with a phenotypic difference thus defines an associated haplotype.

### Example 4

### DNA Pooling: PCR Amplification of Microsatellite Marker, Single Molecule Detection of Pooled DNA Population.

This particular example illustrates the correlation of a different type of information with the phenotypic differences in populations. A microsatellite marker is amplified PCR. The alleles of the amplified microsatellite marker have different lengths. The DNA is then stained with a fluorescent intercalating dye that recognizes a set number of base pairs per intercalator molecule. The population of the amplified microsatellite markers are then introduced into the single molecule analysis system. The different lengths of the DNA are determined using the integrated intensities of the various DNA fragments.

### Example 5

### DNA Pooling, Tagging Using Single Short 6-mer Tag, Analysis of Pooled Population Data for Differences.

The use of a single short 6-mer tag that recognizes multiple sites in a genome allows for recognition of sequences, and also determination of polymorphisms, insertions and deletions. This example illustrates a non-amplified example in which the tagged sequences may represent a small genome. The tagging of the small genome allows the recognition of what differences may be present in the particular experiment. One such experiment may include the recognition of a gene insertion sites in the genome. The correlation of phenotypic differences can then be matched against the site of the inserted gene.

### Example 6

### Reaction Steps in Primer Extension Experiment

The primer extension method is divided into 5 steps. (1) Performing long PCR on DNA from the genomic DNA sample; (2) Denaturing the DNA at 95° C.; (3) Hybridizing the primer products and extending them in the presence of fluorescently labeled dNTPs; (4) Cleaning up the sample using a sephadex spin column; and (5) Introducing the sample into a multi-color single molecule optical reader.

The analysis for the experiment is estimated to be on the order of 3 hours from start to finish. The long PCR step is confirmed in Step 5 of the analysis where the presence of dual color products indicate that both the long PCR product is present and that the haplotype is present as well. Alternatively, the long PCR product can be verified using real-time PCR or gel electrophoresis.

Long PCR can attain DNA lengths up to 23 kilobases in length as demonstrated by Cheng et al., PNAS, 91:5695-5699,1994. An example protocol is as follows (Cheng et al., PNAS, 91:5695-5699, 1994). PCR amplifications (50 μl or 100 μl) were performed in a Perkin-Elmer GeneAmp PCR System 9600, using MicroAmp tubes. All four dNTPs were at 0.2 mM, but other components were varied. For a manual “hot-start,” Mg²⁺ was withheld until samples had been at 75-80° C. for 90 sec and then added from a 25 mM stock. Cycles were as follows: denaturation at 94° C. for 10 sec and annealing and extension at 68° C. for a variable 5-22 min. For times longer than 12-14 min, the autoextension feature was used to add 15-20 sec per cycle, to a final 16-22 min. Depending upon the target copy number and length, 25-38 cycles were used. Most runs (total 6-10 hours) included an initial 10-sec hold at 94° C. and a final 10-min hold at 72° C.

### Example 7

### Experimental Design (Colors, Chip Considerations and Format)

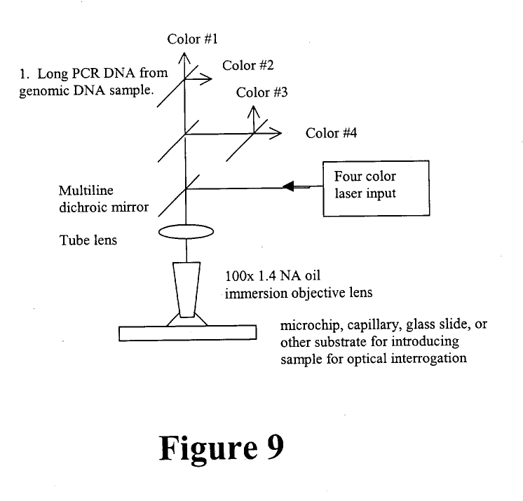

One example of an optical set-up includes a four-color confocal laser-based system (FIG. 9). The laser input is a combination of wavelengths that allows excitation and detection of spectrally separated colors. There are many possible combinations of lasers that can be used for this application. For instance an argon ion laser emits at 488 nm, HeCd laser 441 nm, 405 nm laser, 532 diode laser, 633 HeNe laser, multiline Ar:Kr laser with laser lines in from the UV to the IR. Virtually any laser wavelength is possible through the visible, UV, and IR spectrum. A combination of four laser wavelengths that are compatible with dye chemistries is used for the multicolor excitation of the sample. The laser beams are combined and passed through a four-color dichroic mirror that allows the laser to be reflected at 90° angle through an objective lens that is a 100×1.4NA oil immersion objective. The sample chamber is, for example, a flow-through capillary, glass slide with coverslip, microchip, or other suitable sample chamber that allows handling of the sample. The fluorescence emission from the sample is then directed and captured by the objective lens and passed through multiple dichroics separate the emission into the four spectrally distinct emissions. The signal is captured by fiber-coupled avalanche photodiodes that are at the image plane of the apparatus. The output signal from the APDs is then collected by a computer and analyzed appropriately.

### Example 8

### Use of Multicolor Single-Molecule Detection of PCR Products for Single-Nucleotide Polymorphism and Haplotype Determination

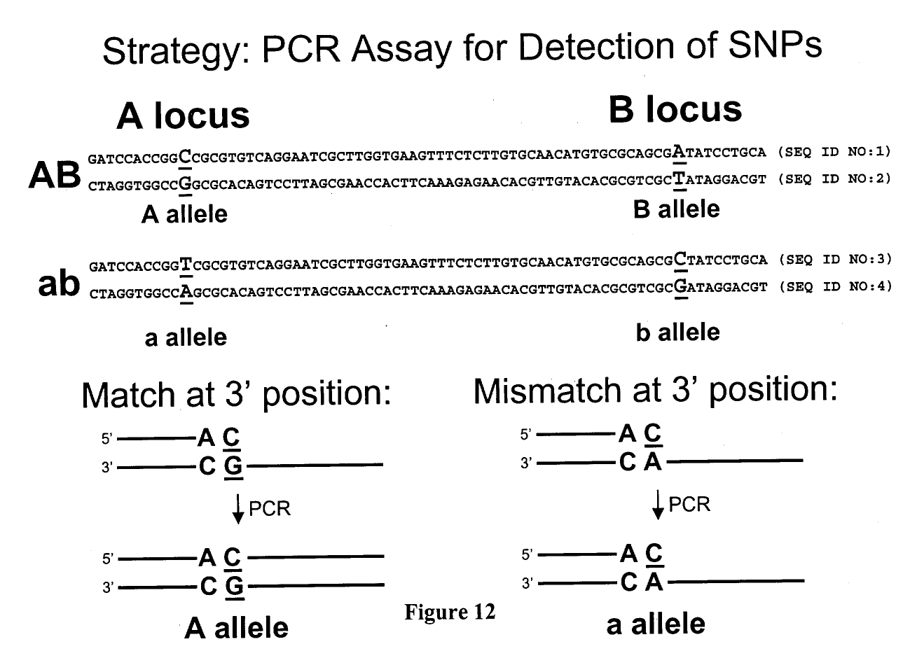

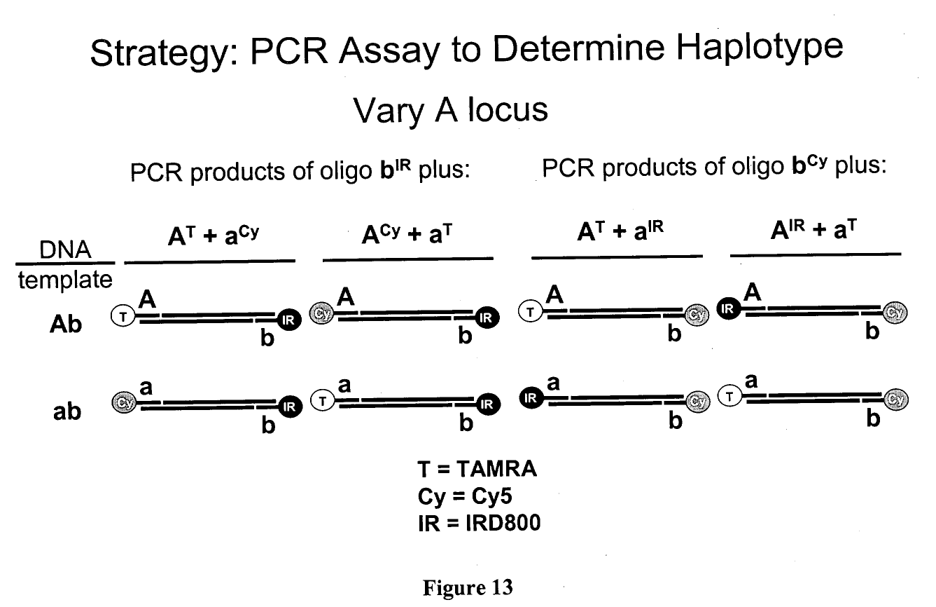

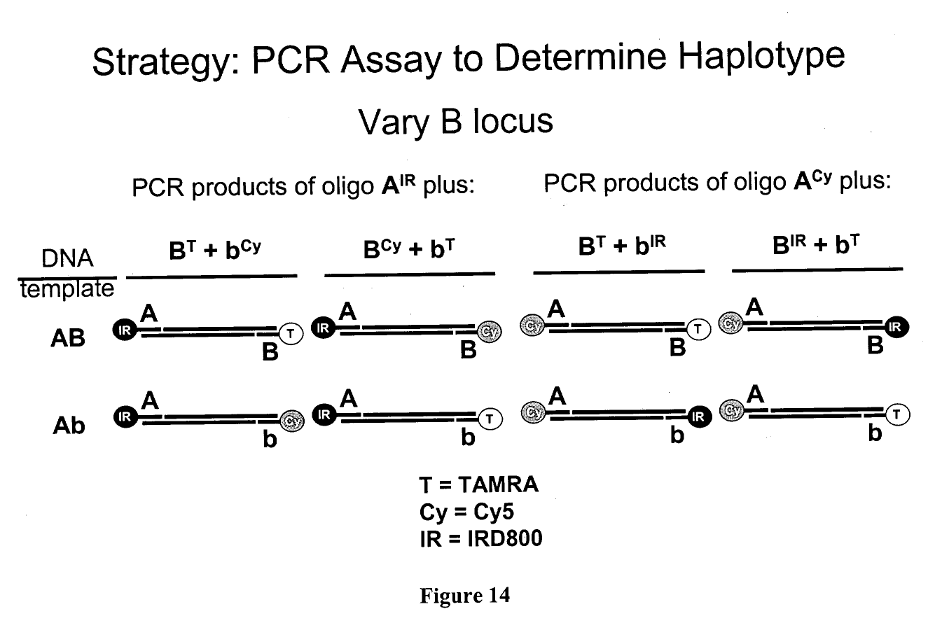

Synthetic DNA templates were constructed and designated AB, Ab, aB and ab (FIG. 12, top). PCR oligos specific for each allele were obtained, where the 3′ end of each oligo contained bases complementary to the cognate sequence and a single base mismatch in the penultimate position (SEQ ID NOs: 1-4, FIG. 12, bottom). Each oligo was chemically synthesized with 5′ ends conjugated to one of three fluorophores, TAMRA, Cy2, or IRD800, via a short tether. Amplification reactions were carried out using combinations of these fluorescent oligos and templates in which the A locus was varied and the B locus was held constant (FIG. 13) or vice versa (FIG. 14).

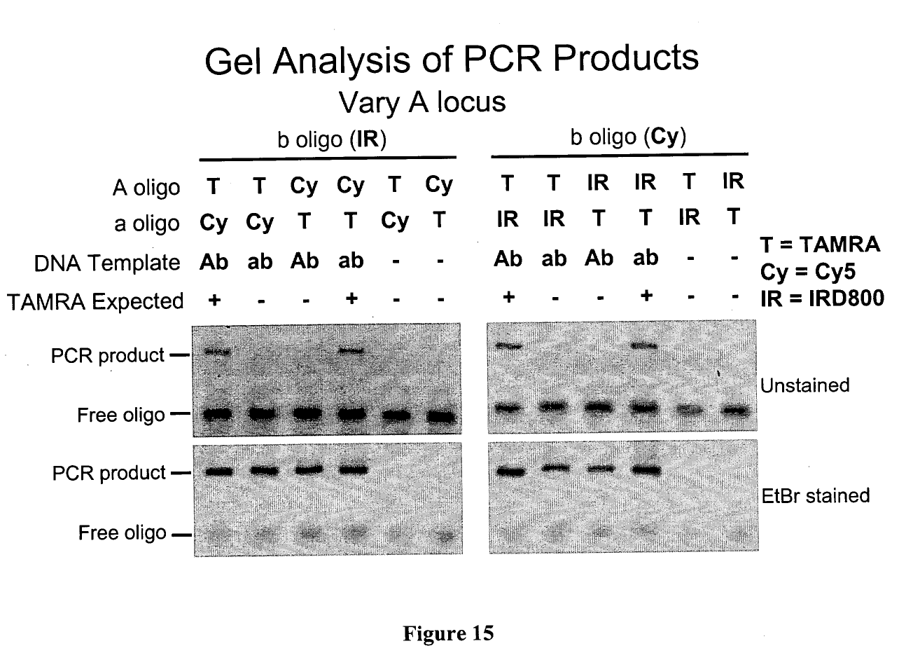

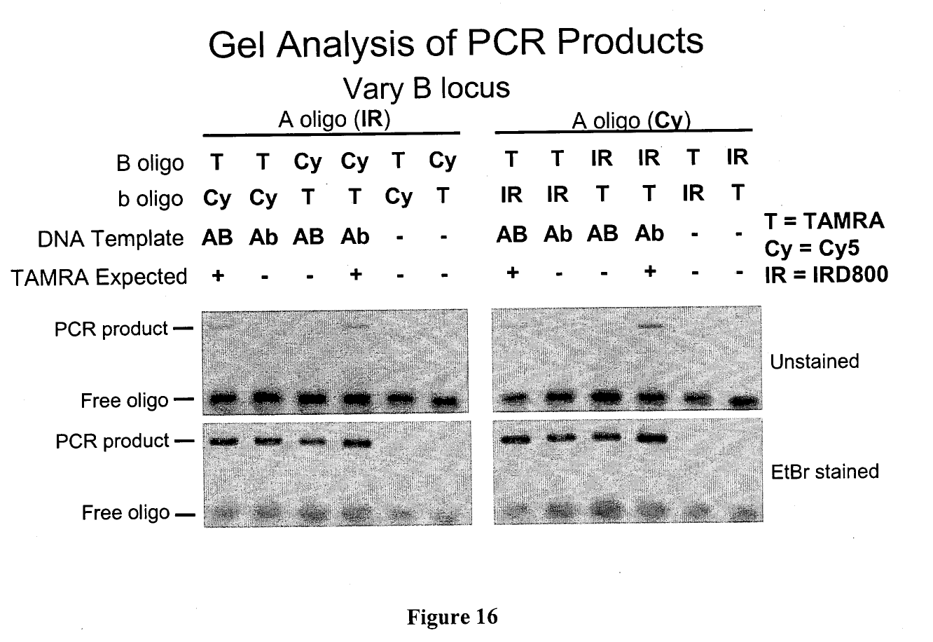

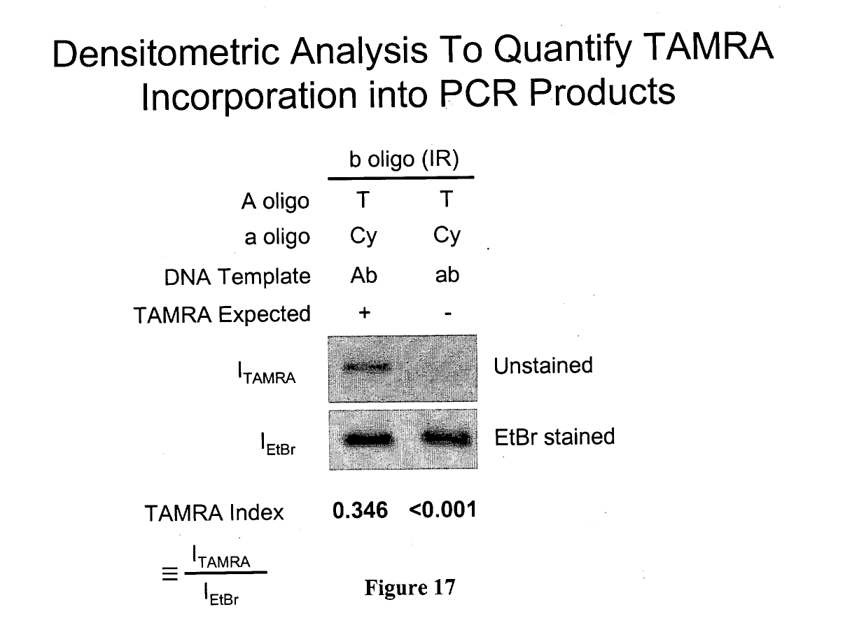

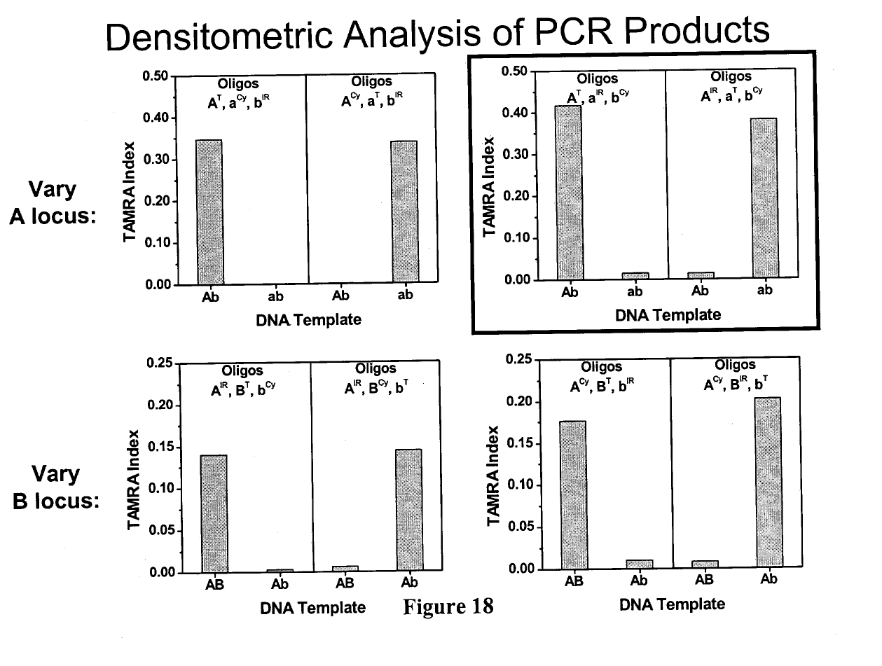

Electrophoretic analysis of the PCR products confirmed the specificity of the oligos for their templates: when the A locus was varied, TAMRA was incorporated into the PCR product only when the 3′ end of the TAMRA-conjugated oligo matched its cognate template (FIG. 15). Similar results were obtained when the B locus was varied (FIG. 16). TAMRA incorporation into the PCR products was quantified using densitometry by dividing the amount of TAMRA intensity by ethidium bromide (EtBr) intensity in the PCR band, as shown in FIG. 17. These TAMRA indices were calculated for each of the discriminatory PCR reactions to quantify the specificity of the PCR reactions (FIG. 18).

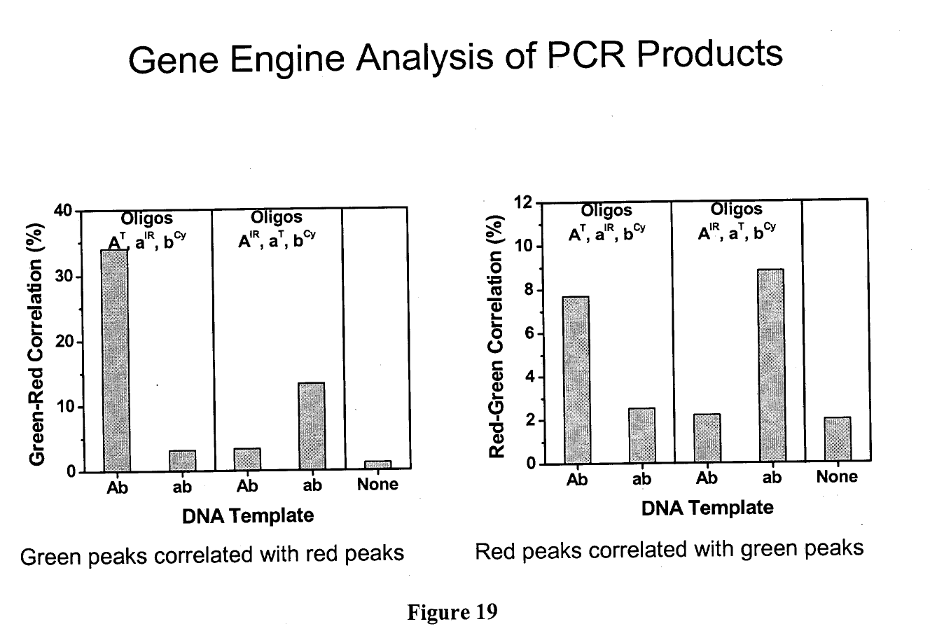

GENEENGINE™ analysis was performed on discriminatory PCR reactions using DNA templates Ab and ab. These reactions contained Cy5 oligos specific for the b allele and TAMRA or IR oligos specific for the A and a alleles (see FIG. 18). Free oligos were removed by passing reaction mixtures over S400 mini spin columns. The correlation of TAMRA with Cy5 (left) or Cy5 with TAMRA (right) was measured (FIG. 19). As expected, correlation was observed in only those PCR reactions in which the TAMRA and Cy5 oligos matched the appropriate alleles. No correlation was observed when the fluorescently-labeled oligos were simply mixed together. This experiment illustrates how the GENEENGINE™ can be used to determine DNA haplotypes, even when only two of its four lasers are utilized.

The assay described above enables the haplotyping of unknown DNA samples in two reactions by detecting a SNP that is linked to a fixed DNA locus. By combining two discriminatory sense oligos specific for distinct alleles in a 5′ locus with two discriminatory anti-sense oligos specific for distinct alleles in a 3′ locus, each of which is conjugated with one of four fluorophores detected, e.g., by the GENEENGINE™, the haplotype of a given DNA sample is determined in a single PCR reaction.

### Equivalents

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. For example, the choice of probe or label type is believed to be a matter of routine for a person of ordinary skill in the art with knowledge of the embodiments described herein. Other aspects, advantages, and modifications considered to be within the scope of the following claims.

We claim:

1. A method of determining a haplotype of a subject, said method comprising providing an extended polynucleotide derived from said subject, said polynucleotide comprising a plurality of target sites that are each similarly labeled with at least a first unit-specific marker and a second unit-specific marker, wherein said at least first unit-specific marker and second unit-specific marker provide information for a haplotype in said subject;
moving the nucleic acid relative to a stationary detection station, and
detecting said plurality of labeled sites at said detection station,
thereby determining a haplotype of a subject.

2. The method of claim 1, wherein the target sites are base sequence variations selected from the group consisting of single nucleotide polymorphism, multibase deletion, multibase insertion, microsatellite repeats, dinucleotide repeats, tri-nucleotide repeats, sequence rearrangements, and chimeric sequence.

3. The method of claim 1, wherein the first and second unit specific markers are luminescent hybridization probes that have a distinguishable characteristic.

4. The method of claim 3, wherein the distinguishable characteristic is selected from the group consisting of luminescence emission spectral distribution, lifetime, intensity, burst duration, and polarization anisotropy.

5. The method of claim 3, wherein the luminescent hybridization probes comprise single dye molecules, energy transfer dye pairs, nano-particles, quantum dots, luminescent nano-crystals, intercalating dyes, or molecular beacons.

6. The method of claim 3, wherein each luminescent hybridization probe specifically hybridizes to one of the plurality of target sites.

7. The method of claim 6, wherein the luminescent hybridization probes are selected from the group consisting of DNA, RNA, locked nucleic acids, and peptide nucleic acids.

8. The method of claim 1, further comprising a third unit-specific marker, wherein said third unit-specific marker provides information for a haplotype in said subject.

9. The method of claim 8, further comprising a fourth unit-specific marker, wherein said fourth unit-specific marker provides information for a haplotype in said subject.

10. The method of claim 1, wherein the unit specific markers are single probes that are specific for each target or multiple probes that act together to identify the target.

11. The method of claim 10, wherein the single probes are selected from the group consisting of oligo DNA, oligo RNA, oligo beacon, oligo peptide nucleic acids, oligo locked nucleic acids, and chimeric oligos.

12. The method of claim 10, wherein the multiple probes are selected from the group consisting of hybridization pairs, invader oligo pairs, ligation oligo pairs, mismatch extension 5′-exonuclease oligo pairs, energy transfer oligo pairs, and 3′-exonuclease pairs.

13. The method of claim 1, wherein the nucleic acid is DNA.

14. The method of claim 13, wherein the nucleic acid is PCR amplified DNA.

15. The method of claim 1, wherein the stationary detection station is in optical communication with an avalanche photo diode or a charge coupled device.

16. The method of claim 14, wherein the nucleic acid is moved through the action of at least one molecular motor.

17. The method of claim 16, wherein the at least one molecular motor is a plurality of molecular motors in solution.

18. The method of claim 14, wherein the nucleic acid is moved through the action of hydrodynamic force.

19. The method of claim 14, wherein the detection station comprises at least one donor fluorophore and wherein a first unit specific marker and a second unit specific marker each comprise at least one acceptor fluorophore.

20. The method of claim 14, wherein the detection station comprises at least one acceptor fluorophore and wherein a first unit specific marker and a second unit specific marker each comprise at least one donor fluorophore.

21. The method of claim 1, wherein the detection station detects fluorescence resonance energy transfer.

22. The method of claim 1, wherein analysis of the nucleic acid provides information about the linear arrangement of target sites within the nucleic acid.

23. The method of claim 1, wherein the detection station detects the plurality of target sites of the nucleic acid simultaneously.

24. The method of claim 23, wherein the unit specific markers are detected by a confocal microscope.

25. The method of claim 23, wherein the plurality of sites are distinguished by labeling each of said plurality of sites with a different colored luminescent hybridization probe.

26. A method of determining a haplotype of a subject comprising moving an extended polynucleotide derived from said subject comprising a plurality of selected genetic markers that are each labeled with at least one distinguishable unit-specific marker, wherein said plurality of selected genetic markers provides information for a haplotype in said subject, through a channel;
exposing said plurality of labeled selected genetic markers to a detection station as the units move relative to the detection station, wherein said plurality of sites interact with the detection station to produce a detectable signal within the channel or at the edge of the channel;
and detecting sequentially the signals resulting from said interaction to analyze the polynucleotide, thereby determining a haplotype of a subject.

27. The method of claim 26, wherein the detection station comprises an agent selected from the group consisting of electromagnetic radiation, a quenching source and a fluorescence excitation source.

28. The method of claim 27, wherein the agent comprises a fluorescence excitation source and said first unit-specific marker and said second unit-specific marker comprise fluorescent hybridization probes.

29. A method for determining a haplotype of a population of nucleic acids in a pool of nucleic acids comprising at least a first population and at least a second population, the method comprising:
providing a pool of extended polynucleotides, wherein the polynucleotides in a population comprises a plurality of target sites that are each similarly labeled with at least a first unit-specific marker and a second unit-specific marker, wherein said at least first unit-specific marker and second unit-specific marker provide information for a haplotype in said pool, further wherein the target sites are selected genetic markers;
moving the polynucleotides of said pool past a stationary detection station;
detecting the luminescent hybridization probes at the stationary detection station; and measuring said luminescent probes as the polynucleotides pass by the detectors, thereby determining the haplotype of the species of the polynucleotides in said pool.

30. The method of claim 29, wherein the target sites are base sequence variations selected from the group consisting of single nucleotide polymorphism, multibase deletion, multibase insertion, microsatellite repeats, dinucleotide repeats, tri-nucleotide repeats, sequence rearrangements, and chimeric sequence.

31. The method of claim 29, wherein the unit specific markers are luminescent hybridization probes that have a distinguishable characteristic.

32. The method of claim 31, wherein the distinguishable characteristic is selected from the group consisting of luminescence emission spectral distribution, lifetime, intensity, burst duration, and polarization anisotropy.

33. The method of claim 31, wherein the luminescent hybridization probes comprise single dye molecules, energy transfer dye pairs, nano-particles, quantum dots, luminescent nano-crystals, intercalating dyes, or molecular beacons.

34. The method of claim 31, wherein each luminescent hybridization probe specifically hybridizes to one of the plurality of target sites.

35. The method of claim 34, wherein the luminescent hybridization probes are selected from the group consisting of DNA, RNA, locked nucleic acids, and peptide nucleic acids.

36. The method of claim 29, further comprising a third unit-specific marker, wherein said third unit-specific marker provides information for a haplotype in said pool.

37. The method of claim 36, further comprising a fourth unit-specific marker, wherein said fourth unit-specific marker provides information for a haplotype in said pool.

38. The method of claim 29, wherein the unit specific markers are single probes that are specific for each target or multiple probes that act together to identify the target.

39. The method of claim 38, wherein the single probes are selected from the group consisting of oligo DNA, oligo RNA, oligo beacon, oligo peptide nucleic acids, oligo locked nucleic acids, and chimeric oligos.

40. The method of claim 38, wherein the multiple probes are selected from the group consisting of hybridization pairs, invader oligo pairs, ligation oligo pairs, mismatch extension 5′-exonuclease oligo pairs, energy transfer oligo pairs, and 3′-exonuclease pairs.

41. The method of claim 29, wherein the polynucleotides are DNA.

42. The method of claim 29, wherein the stationary detection station is in optical communication with an avalanche photo diode or a charge coupled device.

43. The method of claim 29, wherein the detection station detects fluorescence resonance energy transfer.

44. The method of claim 29, wherein the detection station comprises at least one donor fluorophore and wherein a first unit specific marker and a second unit specific marker each comprise at least one acceptor fluorophore.

45. The method of claim 29, wherein the detection station comprises at least one acceptor fluorophore and wherein a first unit specific marker and a second unit specific marker each comprise at least one donor fluorophore.

46. The method of claim 29, wherein the plurality of sites are distinguished by labeling each of said plurality of sites with a different colored luminescent hybridization probe.

47. The method of claim 29, wherein said first population comprises polynucleotides from one individual and said second population comprises polynucleotides from a different individual.

48. The method of claim 29, wherein said first population comprises polynucleotides from a healthy state of a subject and said second population comprises polynucleotides from a disease state of the same subject.

49. A method of determining a haplotype of a subject, said method comprising
providing a polynucleotide, a first ligation oligonucleotide and a second ligation oligonucleotide,
wherein said first ligation oligonucleotide is associated with a first labeled moiety and includes a first constant sequence complementary to a sequence in the target polynucleotide that provides information for a haplotype in said subject, a query nucleotide at the 3′ terminus of said first ligation polynucleotide and, optionally, a mismatch oligonucleotide adjacent to said query nucleotide;
and wherein said second ligation oligonucleotide is associated with a second labeled moiety and includes a second constant sequence complementary to a sequence in the target polynucleotide that provides information for a haplotype in said subject, a query nucleotide at the 3′ terminus of said second ligation polynucleotide and, optionally, a mismatch oligonucleotide adjacent to said query nucleotide;
annealing an effective amount of said first ligation oligonucleotide to said polynucleotide to yield a primed first template;
combining the primed template with an effective amount of a polymerase enzyme and at least two types of nucleotide triphosphates, under conditions sufficient for polymerase activity, thereby forming a first elongated polynucleotide;
annealing an effective amount of said second ligation oligonucleotide to said polynucleotide to yield a primed second template;
combining the primed second template with an effective amount of a polymerase enzyme and at least two types of nucleotide triphosphates, under conditions sufficient for polymerase activity, thereby forming a second elongated polynucleotide;
extending said elongated first polynucleotide and said elongated second polynucleotide; and
detecting said first labeled moiety and second labeled moiety, thereby determining a haplotype.
